(19) United States
(12) Patent Application Publication
(10) Pub. No.: US 2025/0195383 A1
(43) Pub. Date: Jun. 19, 2025

(54) Shampoo Compositions Containing a Sulfate-Free Surfactant System and Sclerotium Gum Thickener

(71) Applicant: The Procter & Gamble Company, Cincinnati, OH (US)

(72) Inventors: Brooke Michele Cochran, Cincinnati, OH (US); Mark Anthony Brown, Union, KY (US); Jean Jianqun Zhao, Cincinnati, OH (US); Lauren Elizabeth Ballhaus, Williamsburg, OH (US); Nicole Marie Mazzei, Cincinnati, OH (US)

(21) Appl. No.: 19/049,671

(22) Filed: Feb. 10, 2025

Related U.S. Application Data
(63) Continuation of application No. 17/738,697, filed on May 6, 2022, now abandoned.
(60) Provisional application No. 63/276,108, filed on Nov. 5, 2021.
(60) Provisional application No. 63/188,517, filed on May 14, 2021.

(51) Int. Cl. A61K 8/60; A61K 8/19; A61K 8/368; A61K 8/49; A61K 8/73; A61K 8/92; A61K 8/9789; A61Q 5/02; A61Q 17/00

(52) U.S. Cl. CPC A61K 8/604; A61K 8/19; A61K 8/368; A61K 8/4926; A61K 8/4946; A61K 8/4953; A61K 8/73; A61K 8/737; A61K 8/922; A61K 8/9789; A61Q 5/02; A61Q 17/005; A61K 2800/33; A61K 2800/5426

(57) ABSTRACT

A stable shampoo composition containing a surfactant system that is substantially free of sulphated surfactants, a cationic polymer, and a sclerotium gum thickener. The shampoo can have a viscosity greater than 2500 cP and a pH greater than 5.5.

### FIELD OF THE INVENTION

The present invention relates to a shampoo composition, in particular a phase-stable shampoo composition with a surfactant system that is free of or substantially free of sulfate-based surfactants and a thickener comprising sclerotium gum.

### BACKGROUND OF THE INVENTION

Historically, most commercial cleansing compositions, such as shampoo compositions, contain sulfate-based surfactant systems because they provide effective cleaning and a good user experience. Sulfate-based surfactant systems generally have acceptable viscosity making it easy to apply and distribute the shampoo composition throughout a user's hair. In addition, sulfate-based surfactant systems can generally be paired with cationic polymers that can form coacervate with the sulfate-based surfactant system during use thereby providing a shampoo with effective conditioning benefits.

Some consumers, especially those with color-treated or otherwise treated hair, may prefer a shampoo composition that is substantially free of sulfate-based surfactant systems. However, it can be difficult to formulate a shampoo with non-sulfate-based surfactants with sufficient viscosity. Many current sulfate-free shampoos have a viscosity that is too low, which makes it difficult to hold in a user's hand and apply across the hair and scalp. These consumers may also want conditioning polymers in their shampoo because higher conditioning shampoos feel less stripping to the hair. However, it can be difficult to formulate a shampoo with a sulfate-free surfactant system that also has cationic polymers that deliver a conditioning benefit because the shampoo can have low viscosity, which makes it difficult for a user to hold the liquid shampoo composition in their palm and distribute across their hair and scalp.

When traditional anionic thickeners, such as xanthan gum, carrageenan, konjac gum, and gellan gum are added to shampoo compositions containing sulfate-free surfactants and cationic polymers, the composition can be unstable and separate into multiple phases. The traditional anionic thickener can complex with the cationic polymer, thereby reducing active deposition, which decreases wet conditioning and causes phase separation.

Therefore, there is a need for a stable shampoo composition with a sufficient viscosity and superior product performance that contains one or more non-sulfated anionic surfactants.

### SUMMARY OF THE INVENTION

A stable shampoo composition comprising: (a) a surfactant system comprising: (i) from about 3 to about 35% of an anionic surfactant; (ii) from about 5 to about 15% of an amphoteric surfactant; wherein the surfactant system is substantially free of sulphated surfactants; (b) from about 0.01% to about 2% of a cationic polymer; (c) from about 0.15% to about 1.5% of a thickener comprising sclerotium gum; wherein the composition comprises a viscosity greater than 2500 cP and a pH greater than 5.5.

### BRIEF DESCRIPTION OF THE DRAWINGS

FIG. 1 shows a typical sclerotium gum structure;

FIG. 2 shows a β-(1,3)-β-(1,6) glucan structure;

FIG. 3 shows a tridimensional conformation of scleroglucan triplex;

FIG. 4 is a 20× micrograph of a shampoo that contains in situ coacervate;

FIG. 5 is 10× micrograph of the shampoo composition of FIG. 1;

FIG. 6A is an image of Comparative Example C5; and

FIG. 6B is an image of Comparative Example C6.

### DETAILED DESCRIPTION OF THE INVENTION

Some consumers prefer shampoo compositions that are substantially free of sulfate-based surfactants. These consumers may also prefer shampoos that feel less stripping to the hair and therefore may prefer a shampoo with cationic conditioning polymers. However, it is difficult to formulate these compositions to be stable and have a consumer preferred viscosity. They are often too thin, which makes them difficult to hold in a user's palm and distribute across their hair and scalp.

There are at least three common ways to thicken shampoo compositions:
1. Add an anionic thickener (e.g. xanthan gum, carrageenan, konjac gum, gellan gum)
2. Decrease the pH
3. Add inorganic salts (e.g. NaCl)

However, these methods are not effective in a shampoo composition that contains sulfate-free anionic surfactants and a cationic conditioning polymer. First, when traditional anionic thickeners are added, it was found that the composition becomes unstable, impacting product appearance and performance. Second, when the pH was decreased it was found that many sulfate-free surfactant systems can hydrolyze at low pH resulting in viscosity and performance changes over time and will eventually lead to phase separation. Third, when inorganic salt was added to the liquid shampoo becomes unstable due to formation of a gel-like complex known as coacervate in the composition (referred to herein as “in situ coacervate” or an “in situ coacervate phase,” which is a coacervate that forms in the composition, prior to dilution), as opposed to when it is diluted with water when a user washes their hair.

It was found that sclerotium gum, which has a unique triple helix structure, could be used to thicken shampoo compositions that contain sulfate-free anionic surfactants and cationic polymers while also maintaining phase stability. It was also found that a thickener comprising sclerotium gum could be used to thicken shampoo compositions that contain sulfate-free surfactants and cationic polymers while also maintaining phase stability and pH (e.g. >5.5), and providing superior wet conditioning.

It was also found that in some examples the amount of inorganic salt in the composition can also impact product stability and viscosity. In some examples, product stability and acceptable viscosity can be achieved by (1) maintaining a low inorganic salt concentration in formulas (e.g. from about 0 to about 1 wt %); and/or (2) having a ratio of polymer charge density to salt of greater than 1.2:1.

One way to lower the inorganic salt concentration is to avoid or minimize adding extra inorganic salt to the formula and/or by using low inorganic salt containing raw materials. For example, commercially available sulfate free surfactants such as disodium cocoyl glutamate typically comes with high levels of inorganic salt such as 5% or higher. Amphoteric surfactant such as betaines or sultaines also typically come with high levels of inorganic salt such as sodium chloride. Use of these high salt containing raw materials in sulfate-free surfactant based cleaning formulations in excess of about 1% total sodium chloride in the formulation can cause formation of undesired coacervate in the product. If the inorganic salt level is lowered in the surfactant raw materials so that total salt in the composition is less than about 1% or lower, a stable 1-phase clear product can be formulated. Whereas, if the regular material with high inorganic salt is used, the product is cloudy, 2-phase, and unstable. The cloudy 2-phase product is likely the result of in situ coacervate formation.

In some examples, sclerotium gum can be the only thickener in the shampoo composition. In other examples, the thickener can include sclerotium gum, inorganic salt, and combinations thereof. The shampoo composition can be free of or substantially free of anionic thickeners. Non-limiting examples of anionic thickeners that may be excluded from the shampoo compositions can include carboxylic acid polymers, crosslinked polyacrylate polymers, polyacrylamide polymers, polysaccharides, and gums. Non-limiting examples of gums can include acacia, agar, algin, alginic acid, ammonium alginate, amylopectin, calcium alginate, calcium carrageenan, carrageenan, dextrin, gelatin, gellan gum, karaya gum, konjac gum, locust bean gum, natto gum, potassium alginate, potassium carrageenan, propylene glycol alginate, sodium carboxymethyl dextran, sodium carrageenan, tragacanth gum, xanthan gum, and mixtures thereof.

The pH can be greater than 5.5, alternatively greater than 5.7, alternatively greater than 5.8. Alternatively, the pH can be from about 5 to about 8, alternatively from about 5.2 to about 7.5, alternatively from about 5.3 to about 7.25, alternatively from about 5.4 to about 7.1, alternatively from about 5.5 to about 7, alternatively from about 5.6 to about 6.9, alternatively from about 5.7 to about 6.8, and alternatively from about 5.8 to about 6.7. The pH can be from about 4 to about 8, alternatively from about 4.5 to about 7.5, alternatively from about 5 to about 7, alternatively from about 5.5 to about 6.5, alternatively from about 5.5 to about 6, and alternatively from about 6 to about 6.5. The pH is determined by the pH Test Method, described herein.

The shampoo composition can have a viscosity greater than 2500 cP, alternatively greater than 4000 cP, alternatively greater than 4500 cP, and alternatively greater than 5000 cP. The shampoo composition can have a viscosity of about 2500 cP to about 20,000 cP, alternatively from about 3000 cps to about 15,000cps, alternatively from 4000 cP to about 13,000 cP, alternatively from about 5,000 cP to about 11,000 cP, and alternatively from about 7,000 cP to about 10,000 cP, alternatively from about 2500 cP to about 8500 cP, alternatively 2500 cP to about 6500 cP as measured at 26.6° C., as measured by the Cone/Plate Viscosity Measurement Test Method, described herein.

The shampoo composition can have a yield stress, Herschel-Bulkley @ shear rate 10⁻² to 10⁻⁴ s⁻¹ of from about 0.001 Pa to about 0.5 Pa, alternatively from about 0.002 Pa to about 0.3 Pa, alternatively from about 0.003 Pa to about 0.2 Pa, alternatively from about 0.004 Pa to about 0.18Pa, alternatively from about 0.007 Pa to about 0.17 Pa, and alternatively from about 0.008 Pa to about 0.15 Pa. The shampoo composition can have a yield stress, Herschel-Bulkley @ shear rate 10⁻² to 10⁻⁴ s⁻¹ of greater than 0.003 Pa, alternatively greater than 0.005 Pa, alternatively greater than 0.010 Pa, alternatively greater than 0.015 Pa, and alternatively greater than 0.1 Pa. The yield stress is measured at 26.7° C. by flow sweep at a shear rate 100 to 10⁻⁴ s⁻¹ using Discovery Hybrid Rheometer (DHR-3) available from TA Instruments. To apply the Hershel-Bulkley model, the TA software to fit the model in the log space at a shear rate from 10⁻² to 10⁻⁴ s⁻¹ is used. The geometry used to measure the yield stress and viscosity of the cleansing phase is a 60 mm 2° aluminum cone (with a Peltier steel plate). The geometry should be run at the gap specified by the manufacturer for the geometry. Trimming the sample during the initial conditioning step in step 1 is recommended to ensure data integrity and reproducibility. Torque map the geometry prior to running the yield stress or shear stress methods when the instrument+geometry is out of calibration. The version of Trios software used to generate the rheology data herein is TRIOS 5.1.1

In some examples, it was found that a thickener that contained a combination of sclerotium gum and citrus peel fiber (commercially available as FiberDesign™ Sensation from Cargill® with an INCI citrus limon peel powder (and) sclerotium gum) the shampoo composition could have a higher yield stress, which can help suspend actives (e.g. cationic polymers; silicones; non-silicone organic conditioning agents such as botanical hydrocarbon oils, waxes, conditioning polymers; anti-dandruff actives) in the formulation, which can lead to improved product performance. In some examples the thickener can be selected from the group consisting of sclerotium gum, citrus limon peel powder, inorganic salts, and combinations thereof. In other examples the thickener can be selected from the group consisting of sclerotium gum, citrus limon peel powder, and combinations thereof. In other examples, the thickener can contain both sclerotium gum and citrus limon peel powder.

The shampoo composition may have less than 1% of inorganic salt, alternatively less than 0.75% inorganic salt, alternatively less than 0.5% inorganic salt, alternatively less than 0.25% inorganic salt, alternatively less than or equal to 0.2% inorganic salt. The shampoo composition may have from about 0% to about 1% inorganic salt, alternatively from about 0% to about 0.9% inorganic salt, alternatively from about 0% to about 0.8%, alternatively from about 0% to about 0.5% inorganic salt, alternatively from about 0% to about 0.3% inorganic salt; and alternatively from about 0.05% to about 0.2% inorganic salt. The shampoo composition may have greater than 0% and less than 1% inorganic salt, alternatively greater than 0% and less than 0.75%, alternatively greater than 0% and less than 0.5%, and alternatively greater than 0% and less than 0.25%. The shampoo composition can include from about 0.5% to about 5% inorganic salt, alternatively from about 0.55% to about 4%, alternatively about 0.75% to about 3.5%, alternatively 0.6% to about 3.25%, alternatively from about 0.8% to about 3%, alternatively from about 0.8% to about 2.5%, alternatively from about 1% to about 2%, and alternatively from about 1% to about 1.5%. The shampoo composition can include from about 0.75% to about 1.5% inorganic salt, alternatively from about 0.8% to about 1.4%, alternatively from about 0.9% to about 1.4%. The wt. % inorganic chloride salt can be determined by the Argentometry Method to Measure wt % Inorganic Chloride Salt Test Method, described herein. The inorganic salt can be an inorganic chloride salt. The inorganic salt can enter the composition with the surfactants or other ingredients, or it can be added separately as a thickener. If the inorganic salt enters the composition along with the surfactants, it can still considered a thickener.

The ratio of polymer charge density to total inorganic salt can be from about 0.5:1 to about 3:1, from about 0.7:1 to about 2.5:1, alternatively 0.75:1 to about 2.25:1, alternatively from about 1:1 to about 2:1, alternatively from about 1.2:1 to about 1.5:1, alternatively from about 1.1:1 to about 1.4:1, and alternatively from about 1.2:1 to about 1.4:1. The ratio of polymer charge density to total inorganic salt can be ≥1.1:1, alternatively ≥1.2:1, alternatively ≥1.3:1, and alternatively ≥1.5:1. The ratio of polymer charge density to total inorganic salt can be <15:1, <10:1, <7:1, <5:1, alternatively <4:1, alternatively <3:1, alternatively <2:1, and alternatively <1.5:1. The ratio of polymer charge density to inorganic salt is the charge density of the cationic polymer (meq/gm) to the wt. % of the inorganic salt, disregarding the units. If the composition contains more than one cationic polymer, then the ratio is calculated according to the polymer with the lowest charge density.

The ratio of anionic surfactant to amphoteric surfactant can be from about 0.25:1 to about 3:1, alternatively from about 0.3:1 to about 2.5:1, alternatively from about 0.4:1 to about 2:1, alternatively from about 0.5:1 to about 1.5:1, alternatively from about 0.6:1 to about 1.25:1, and alternatively from about 0.75:1 to about 1:1. The ratio of anionic surfactant to amphoteric surfactant can be from about 0.4:1 to about 1.25:1, alternatively from about 0.5:1 to about 1.1:1, and alternatively from about 0.6:1 to about 1:1. In some examples, the ratio of anionic surfactant to amphoteric surfactant is less than 2:1, alternatively less than 1.75:1, alternatively less than 1.5:1, alternatively less than 1.1:1, and alternatively less than 1:1.

The shampoo composition can be used to clean and/or condition hair. First, the user dispenses the liquid shampoo composition from the bottle into their hand or onto a cleaning implement. Then, they massage the shampoo into their wet hair. While they are massaging the shampoo composition into the hair the shampoo is diluted and a coacervate can form and the shampoo can lather. After massaging into hair, the shampoo composition is rinsed from the user's hair and at least a portion of the cationic polymers can be deposited on the user's hair, which can provide a wet conditioning benefit. Shampooing can be repeated, if desired, and/or a conditioner can be applied. The conditioner can be a rinse-off conditioner or a leave-in conditioner.

It may be consumer desirable to have a shampoo composition with a minimal level of ingredients. The shampoo composition can be formulated without polymeric thickeners or suspending agents such as carbomer, EGDS or thixcin. The shampoo composition may contain 11 or fewer ingredients, 10 or fewer ingredients, 9 or fewer ingredients, 8 or fewer ingredients, 7 or fewer ingredients, 6 or fewer ingredients. The minimal ingredient formula can include water, anionic surfactant, amphoteric surfactant, cationic polymer, inorganic salt, and perfume. It is understood that perfumes can be formed from one or more materials. In some examples, the composition can be free of or substantially free of fragrance. In another example, the composition can be free of or substantially free of polyethylene glycol (PEG).

The composition can be free of alkyl polyglucoside. Non-limiting examples of alkyl polyglucosides can include decyl glucoside, cocoyl glucoside, lauroyl glucoside and combinations thereof. It has been found that alky polyglucosides can impact lather properties and thickening of shampoo compositions containing sulfate-free anionic surfactants and cationic conditioning polymers.

The composition can be substantially free of or free of fatty esters having at least 10 carbon atoms. The composition can be substantially free of or free of fatty esters. The composition can be substantially free of or free of fatty esters with hydrocarbyl chains derived from fatty acids or alcohols. The composition can be substantially free of or free of oligomeric or polymeric esters, prepared from unsaturated glyceryl esters can also be used as conditioning materials.

While the specification concludes with claims particularly pointing out and distinctly claiming the invention, it is believed that the present disclosure will be better understood from the description.

As used herein, “cleansing composition” includes personal cleansing products such as shampoos, conditioners, conditioning shampoos, shower gels, liquid hand cleansers, facial cleansers, and other surfactant-based liquid compositions.

The shampoo composition can be clear prior to dilution with water. The term “clear” or “transparent” as used herein, means that the compositions have a percent transparency (% T) of at least about 70% transmittance at 600 nm. The % T may be at 600 nm from about 70% to about 100%, from about 75% to about 100%, from about 80% to about 100%, from about 85% to about 100%, from about 90% to about 100%, from about 95% to about 100%. In the present invention, the percent transparency (% T) may be at least about 80% transmittance at 600 nm; percent transparency (% T) may be at least about 90% transmittance at 600 nm.

As used herein, the term “fluid” includes liquids and gels.

As used herein, “molecular weight” or “M.Wt.” refers to the weight average molecular weight unless otherwise stated. Molecular weight is measured using industry standard method, gel permeation chromatography (“GPC”). The molecular weight has units of grams/mol.

As used herein, “substantially free” refers to less than 0.5%, alternatively less than 0.25%, alternatively less than 0.1%, alternatively less than 0.05%, alternatively less than 0.02%, and alternatively less than 0.01%.

As used herein, “sulfate free” and “substantially free of sulfates” means essentially free of sulfate-containing compounds except as otherwise incidentally incorporated as minor components. Sulfate free contains no detectable sulfated surfactants.

As used herein, “sulfated surfactants” or “sulfate-based surfactants” means surfactants which contain a sulfate group. The term “substantially free of sulfated surfactants” or “substantially free of sulfate-based surfactants” means essentially free of surfactants containing a sulfate group except as otherwise incidentally incorporated as minor components.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Surfactant

The cleansing compositions described herein can include one or more surfactants in the surfactant system. The one or more surfactants can be substantially free of sulfate-based surfactants. As can be appreciated, surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. Suitable surfactants for a cleansing composition can include anionic moieties to allow for the formation of a coacervate with a cationic polymer. The surfactant can be selected from anionic surfactants, amphoteric surfactants, zwitterionic surfactants, non-ionic surfactants, and combinations thereof.

Cleansing compositions typically employ sulfate-based surfactant systems (such as, but not limited to, sodium lauryl sulfate) because of their effectiveness in lather production, stability, clarity and cleansing. The cleansing compositions described herein are substantially free of sulfate-based surfactants. “Substantially free” of sulfate based surfactants as used herein means from about 0% to about 3%, alternatively from about 0% to about 2%, alternatively from about 0% to about 1%, alternatively from about 0% to about 0.5%, alternatively from about 0% to about 0.25%, alternatively from about 0% to about 0.1%, alternatively from about 0% to about 0.05%, alternatively from about 0% to about 0.01%, alternatively from about 0% to about 0.001%, and/or alternatively free of sulfates. As used herein, “free of” means 0%.

Additionally, the surfactants can be added to the composition as a solution, instead of the neat material and the solution can include inorganic salts that can be added to the formula. The surfactant formula can have inorganic salt that can be from about 0% to about 2% of inorganic salts of the final composition, alternatively from about 0.1% to about 1.5%, and alternatively from about 0.2% to about 1%.

Suitable surfactants that are substantially free of sulfates can include sodium, ammonium or potassium salts of isethionates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

The concentration of the surfactant in the composition should be sufficient to provide the desired cleaning and lather performance. The cleansing composition can include a total surfactant level of from about 5% to about 50%, alternatively from about 8% to about 40%, alternatively from about 10% to about 30%, alternatively from about 12% to about 25%, alternatively from about 13% to about 23%, alternatively from about 14% to about 21%, alternatively from about 15% to about 20%.

The cleansing composition can include from about 3% to about 30% anionic surfactant, alternatively from about 4% to about 20%, alternatively from about 5% to about 15%, alternatively from about 6% to about 12%, and alternatively from about 7% to about 10%. The cleansing composition can include from about 3% to about 40% amphoteric surfactant, alternatively from about 4% to about 30%, alternatively from about 5% to about 25%, alternatively from about 6% to about 18%, alternatively from about 7% to about 15%, alternatively from about 8% to about 13%, and alternatively from about 9% to about 11%.

The ratio of anionic surfactant to amphoteric surfactant can be from about 0.25:1 to about 3:1, alternatively from about 0.3:1 to about 2.5:1, alternatively from about 0.4:1 to about 2:1, alternatively from about 0.5:1 to about 1.5:1, alternatively from about 0.6:1 to about 1.25:1, and alternatively from about 0.75:1 to about 1:1. The ratio of anionic surfactant to amphoteric surfactant can be from about 0.4:1 to about 1.25:1, alternatively from about 0.5:1 to about 1.1:1, and alternatively from about 0.6:1 to about 1:1. In some examples, the ratio of anionic surfactant to amphoteric surfactant is less than 2:1, alternatively less than 1.75:1, alternatively less than 1.5:1, alternatively less than 1.1:1, and alternatively less than 1:1.

In some examples, inorganic salt is added to the shampoo composition with the surfactant raw materials. In one example, the surfactant raw materials include less than 1.5% inorganic salt, alternatively less than 1.25%, alternatively less than 1%, alternatively less than 0.7%, alternatively less than 0.5%, alternatively less than 0.25%, alternatively less than 0.2%, alternatively less than 0.15%, alternatively less than or equal to 0.1%. In some examples, at least 0.05% inorganic salt is added to the formula via the surfactant raw materials, alternatively at least 0.07%, and alternatively at least 0.1%.

The surfactant system can include one or more amino acid based anionic surfactants. Non-limiting examples of amino acid based anionic surfactants can include sodium, ammonium or potassium salts of acyl glycinates; sodium, ammonium or potassium salts of acyl sarcosinates; sodium, ammonium or potassium salts of acyl glutamates; sodium, ammonium or potassium salts of acyl alaninates and combinations thereof.

The amino acid based anionic surfactant can be a glutamate, for instance an acyl glutamate. Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl Glutamate, sodium olivoyl glutamate, disodium olivoyl glutamate, sodium palmoyl glutamate, disodium palmoyl Glutamate, TEA-cocoyl glutamate, TEA-hydrogenated tallowoyl glutamate, TEA-lauroyl glutamate, and mixtures thereof.

The amino acid based anionic surfactant can be an alaninate, for instance an acyl alaninate. Non-limiting example of acyl alaninates can include sodium cocoyl alaninate, sodium lauroyl alaninate, sodium N-dodecanoyl-1-alaninate and combination thereof.

The amino acid based anionic surfactant can be a sarcosinate, for instance an acyl sarcosinate. Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

The amino acid based anionic surfactant can be a glycinate for instance an acyl glycinate. Non-limiting example of acyl glycinates can include sodium cocoyl glycinate, sodium lauroyl glycinate and combination thereof.

The composition can contain additional anionic surfactants selected from the group consisting of sulfosuccinates, isethionates, sulfonates, sulfoacetates, glucose carboxylates, alkyl ether carboxylates, acyl taurates, and mixture thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinnate, diamyl ester of sodium sulfosuccinic acid, dihexyl ester of sodium sulfosuccinic acid, dioctyl esters of sodium sulfosuccinic acid, and combinations thereof. The composition can comprise a sulfosuccinate level from about 2% to about 22%, by weight, from about 3% to about 19%, by weight, 4% to about 17%, by weight, and/or from about 5% to about 15%, by weight. Suitable isethionate surfactants can include the reaction product of fatty acids esterified with isethionic acid and neutralized with sodium hydroxide. Suitable fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, and mixtures thereof.

Non-limiting examples of sulfonates can include alpha olefin sulfonates, linear alkylbenzene sulfonates, sodium laurylglucosides hydroxypropylsulfonate and combination thereof.

Non-limiting examples of sulfoacetates can include sodium lauryl sulfoacetate, ammonium lauryl sulfoacetate and combination thereof.

Non-limiting example of glucose carboxylates can include sodium lauryl glucoside carboxylate, sodium cocoyl glucoside carboxylate and combinations thereof.

Non-limiting example of alkyl ether carboxylate can include sodium laureth-4 carboxylate, laureth-5 carboxylate, laureth-13 carboxylate, sodium C12-13 pareth-8 carboxylate, sodium C12-15 pareth-8 carboxylate and combination thereof.

Non-limiting example of acyl taurates can include sodium methyl cocoyl taurate, sodium methyl lauroyl taurate, sodium methyl oleoyl taurate, sodium caproyl methyltaurate and combination thereof.

The surfactant system may further comprise one or more amphoteric surfactants and the amphoteric surfactant can be selected from the group consisting of betaines, sultaines, hydroxysultanes, amphohydroxypropyl sulfonates, alkyl amphoactates, alkyl amphodiacetates, alkyl amphopropionates and combination thereof.

Examples of betaine amphoteric surfactants can include coco dimethyl carboxymethyl betaine, cocoamidopropyl betaine (CAPB), cocobetaine, lauryl amidopropyl betaine (LAPB), oleyl betaine, coco-betaine, cetyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, and mixtures thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

Non-limiting example of alkylamphoacetates can include sodium cocoyl amphoacetate, sodium lauroyl amphoacetate and combination thereof.

The amphoteric surfactant can comprise cocamidopropyl betaine (CAPB), lauramidopropyl betaine (LAPB), and combinations thereof.

The surfactant system may further comprise one or more non-ionic surfactants and the non-ionic surfactant can be selected from the group consisting alkyl polyglucoside, alkyl glycoside, acyl glucamide and mixture thereof. Non-limiting examples of alkyl polyglucosides can include decyl glucoside, cocoyl glucoside, lauroyl glucoside and combination thereof.

Non-limiting examples of acyl glucamide can include lauroyl/myristoyl methyl glucamide, capryloyl/caproyl methyl glucamide, lauroyl/myristoyl methyl glucamide, cocoyl methyl glucamide and combinations thereof.

The composition can contain a non-ionic detersive surfactants that can include cocamide, cocamide methyl MEA, cocamide DEA, cocamide MEA, cocamide MIPA, lauramide DEA, lauramide MEA, lauramide MIPA, myristamide DEA, myristamide MEA, PEG-20 cocamide MEA, PEG-2 cocamide, PEG-3 cocamide, PEG-4 cocamide, PEG-5 cocamide, PEG-6 cocamide, PEG-7 cocamide, PEG-3 lauramide, PEG-5 lauramide, PEG-3 oleamide, PPG-2 cocamide, PPG-2 hydroxyethyl cocamide, and mixtures thereof.

### Sclerotium Gum

The shampoo composition comprises 0.15 wt % to 1.05 wt % sclerotium gum, 0.15 wt % to 1.0 wt % sclerotium gum, 0.2 wt % to 0.8 wt % sclerotium gum, 0.4 wt % to 0.8 wt % sclerotium gum, and/or 0.4 wt % to 0.6 wt % sclerotium gum, and any combination thereof. Sclerotium gum is also called scleroglucan, and it is a branched polysaccharide. In some instances, the primary structure of the scleroglucan consists of glucose molecules linked by β-(1,3) linkage, and every third glucose molecule in the primary structure contains an additional glucose molecule linked by a β-(1,6) linkage. In certain solutions, scleroglucan forms a triple helix shape.

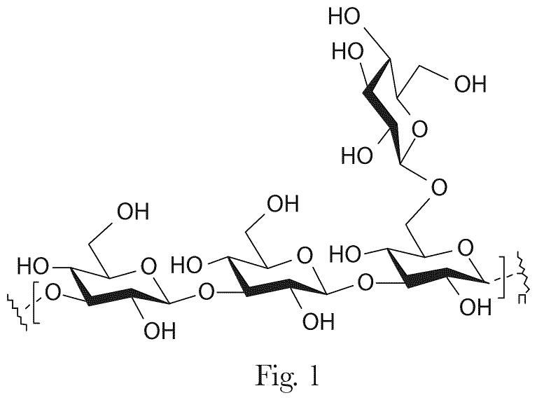

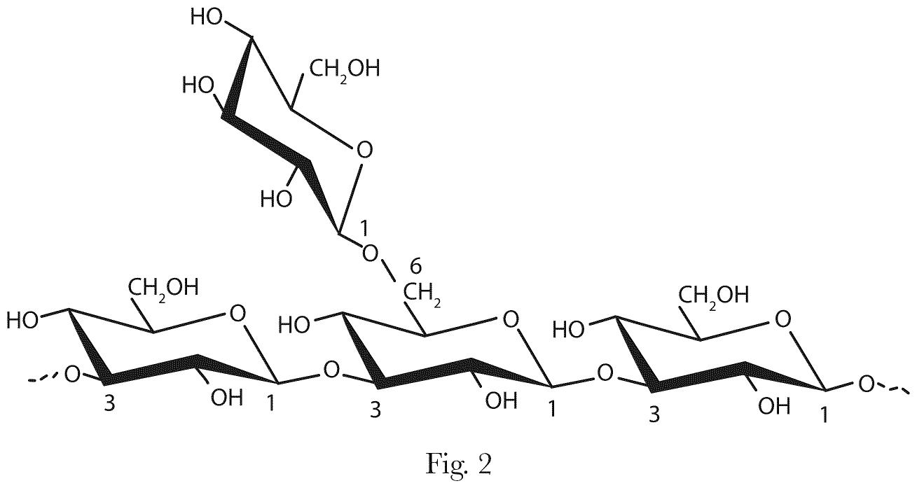

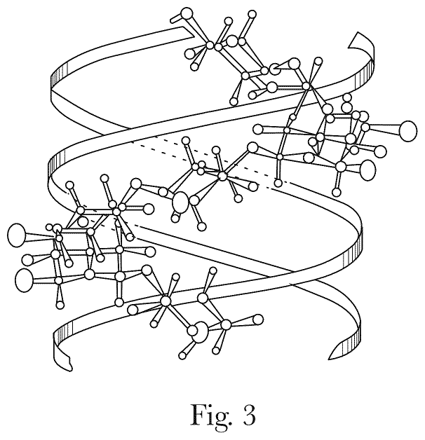

FIG. 1 shows an example of a typical sclerotium gum structure. FIG. 2 shows an example of a β-(1,3)-β-(1,6) glucan structure exhibiting the (3:1) side branching ratio of scleroglucan (Martin et al., 2007). FIG. 3 shows an example of a tridimensional conformation of a scleroglucan triplex (Crescenzi et al., 1988). Specific examples of sclerotium gum include Amigum ER commercially available from Alban Muller, Actigum CS 11 QD commercially available from Cargill.

### Cationic Polymer

A cleansing composition can include a cationic polymer to allow formation of a coacervate. As can be appreciated, the cationic charge of a cationic polymer can interact with an anionic charge of a surfactant to form the coacervate. Suitable cationic polymers can include: (a) a cationic guar polymer, (b) a cationic non-guar galactomannan polymer, (c) a cationic starch polymer, (d) a cationic copolymer of acrylamide monomers and cationic monomers, (e) a synthetic, non-crosslinked, cationic polymer, which may or may not form lyotropic liquid crystals upon combination with the detersive surfactant, and (f) a cationic cellulose polymer. In certain examples, more than one cationic polymer can be included.

A cationic polymer can be included by weight of the cleansing composition at about 0.05% to about 3%, about 0.075% to about 2.0%, or at about 0.1% to about 1.0%. Cationic polymers can have cationic charge densities of about 0.6 meq/g or more, of about 0.9 meq/g or more, about 1.2 meq/g or more, and about 1.5 meq/g or more. However, cationic charge density can also be about 7 meq/g or less and alternatively about 5 meq/g or less. Cationic polymers can have cationic charge densities of from about 0.2 meq/g to about 2.2 meq/g, from about 0.3 meq/g to about 2.0 meq/g, from about 0.4 meq/g to about 1.8 meq/g; from about 0.5 meq/g to about 1.7 meq/g and from about 0.6 meq/g to about 1.3. In some examples the composition can include a cationic polymer with charge density of about 1.7 to about 2.1 meq/g and about 1 to about 1.5% total inorganic salt. The charge densities can be measured at the pH of intended use of the cleansing composition. (e.g., at about pH 3 to about pH 9; or about pH 4 to about pH 8). The average molecular weight of cationic polymers can generally be between about 10,000 and 10 million, between about 50,000 and about 5 million, and between about 100,000 and about 3 million, and between about 300,000 and about 3 million and between about 100,000 and about 2.5 million. Low molecular weight cationic polymers can be used. Low molecular weight cationic polymers can have greater translucency in the liquid carrier of a cleansing composition. The cationic polymer can be a single type, such as the cationic guar polymer guar hydroxypropyltrimonium chloride having a weight average molecular weight of about 2.5 million g/mol or less, and the cleansing composition can have an additional cationic polymer of the same or different types.

Charge density of cationic polymers other than cationic guar polymers can be determined by measuring % Nitrogen. % Nitrogen is measured using USP <461> Method II. % Nitrogen can then be converted to Cationic Polymer Charge Density by calculations known in the art.

The charge density of cationic guar polymers can be calculated as follows: first, calculate the degree of substitution, as disclosed in WO 2019/096601, page 3, lines 4-22, and then cationic charge density can be calculated from the degree of substitution, as described in WO 2013/011122, page 8, lines 8-17, the disclosure of these publications are incorporated by reference.

### Cationic Guar Polymer

The cationic polymer can be a cationic guar polymer, which is a cationically substituted galactomannan (guar) gum derivative. Suitable guar gums for guar gum derivatives can be obtained as a naturally occurring material from the seeds of the guar plant. As can be appreciated, the guar molecule is a straight chain mannan which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums can be obtained through reactions between the hydroxyl groups of the polygalactomannan and reactive quaternary ammonium compounds. The degree of substitution of the cationic groups onto the guar structure can be sufficient to provide the requisite cationic charge density described above.

A cationic guar polymer can have a weight average molecular weight (“M.Wt.”) of less than about 3 million g/mol, and can have a charge density from about 0.05 meq/g to about 2.5 meq/g. Alternatively, the cationic guar polymer can have a weight average M.Wt. of less than 1.5 million g/mol, from about 150 thousand g/mol to about 1.5 million g/mol, from about 200 thousand g/mol to about 1.5 million g/mol, from about 300 thousand g/mol to about 1.5 million g/mol, and from about 700,000 thousand g/mol to about 1.5 million g/mol. The cationic guar polymer can have a charge density from about 0.2 meq/g to about 2.2 meq/g, from about 0.3 meq/g to about 2.0 meq/g, from about 0.4 meq/g to about 1.8 meq/g, from about 0.5 meq/g to about 1.7 meq/g, and from about 0.6 meq/g to about 1.3 meq/g.

A cationic guar polymer can have a weight average M.Wt. of less than about 1 million g/mol, and can have a charge density from about 0.1 meq/g to about 2.5 meq/g. A cationic guar polymer can have a weight average M.Wt. of less than 900 thousand g/mol, from about 150 thousand to about 800 thousand g/mol, from about 200 thousand g/mol to about 700 thousand g/mol, from about 300 thousand to about 700 thousand g/mol, from about 400 thousand to about 600 thousand g/mol, from about 150 thousand g/mol to about 800 thousand g/mol, from about 200 thousand g/mol to about 700 thousand g/mol, from about 300 thousand g/mol to about 700 thousand g/mol, and from about 400 thousand g/mol to about 600 thousand g/mol. A cationic guar polymer has a charge density from about 0.2 meq/g to about 2.2 meq/g, from about 0.3 meq/g to about 2.0 meq/g, from about 0.4 meq/g to about 1.8 meq/g; and from about 0.5 meq/g to about 1.5 meq/g.

A cleansing composition can include from about 0.01% to less than about 0.7%, by weight of the cleansing composition of a cationic guar polymer, from about 0.04% to about 0.55%, by weight, from about 0.08% to about 0.5%, by weight, from about 0.16% to about 0.5%, by weight, from about 0.2% to about 0.5%, by weight, from about 0.3% to about 0.5%, by weight, and from about 0.4% to about 0.5%, by weight.

The cationic guar polymer can be formed from quaternary ammonium compounds which conform to general Formula II:

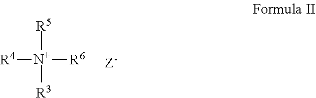

wherein where R³, R⁴ and R⁵ are methyl or ethyl groups; and R⁶ is either an epoxyalkyl group of the general Formula III:

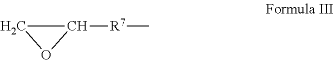

or R⁶ is a halohydrin group of the general Formula IV:

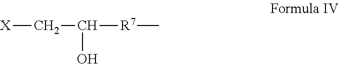

wherein R⁷ is a C₁ to C₃ alkylene; X is chlorine or bromine, and Z is an anion such as Cl—, Br—, I— or HSO₄—.
Suitable cationic guar polymers can conform to the general formula V:

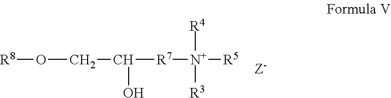

wherein R⁸ is guar gum; and wherein R⁴, R⁵, R⁶ and R⁷ are as defined above; and wherein Z is a halogen. Suitable cationic guar polymers can conform to Formula VI:

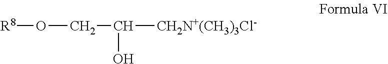

wherein R⁸ is guar gum.

Suitable cationic guar polymers can also include cationic guar gum derivatives, such as guar hydroxypropyltrimonium chloride. Suitable examples of guar hydroxypropyltrimonium chlorides can include the Jaguar® series commercially available from Solvay® S.A., Hi-Care Series from Rhodia®, and N-Hance™ and AquaCat™ from Ashland™ Inc. For example, N-Hance™ BF-17 is a borate (boron) free guar polymers. N-Hance™ BF-17 has a charge density of about 1.7 meq/g and M.Wt. of about 800,000. BF-17 has a charge density of about 1.7 meq/g and M.Wt. of about 800,000. BF-17 has a charge density of about 1.7 meq/g and M.Wt. of about 800,000. BF-17 has a charge density of about 1.7 meq/g and M.Wt. of about 800,000. BF-17 has a charge density of about 1.7 meq/g and M.Wt. of about 800,000.

### Cationic Non-Guar Galactomannan Polymer

The cationic polymer can be a galactomannan polymer derivative. Suitable galactomannan polymer can have a mannose to galactose ratio of greater than 2:1 on a monomer to monomer basis and can be a cationic galactomannan polymer derivative or an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers can be present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β(1-4) glycosidic linkages. The galactose branching arises by way of an α(1-6) linkage. The ratio of mannose monomers to galactose monomers varies according to the species of the plant and can be affected by climate. Non Guar Galactomannan polymer derivatives can have a ratio of mannose to galactose of greater than 2:1 on a monomer to monomer basis. Suitable ratios of mannose to galactose can also be greater than 3:1 or greater than 4:1. Analysis of mannose to galactose ratios is well known in the art and is typically based on the measurement of the galactose content.

The gum for use in preparing the non-guar galactomannan polymer derivatives can be obtained from naturally occurring materials such as seeds or beans from plants. Examples of various non-guar galactomannan polymers include Tara gum (3 parts mannose/1 part galactose), Locust bean or Carob (4 parts mannose/1 part galactose), and Cassia gum (5 parts mannose/1 part galactose).

A non-guar galactomannan polymer derivative can have a M. Wt. from about 1,000 g/mol to about 10,000,000 g/mol, and a M.Wt. from about 5,000 g/mol to about 3,000,000 g/mol.

The cleansing compositions described herein can include galactomannan polymer derivatives which have a cationic charge density from about 0.5 meq/g to about 7 meq/g. The galactomannan polymer derivatives can have a cationic charge density from about 1 meq/g to about 5 meq/g. The degree of substitution of the cationic groups onto the galactomannan structure can be sufficient to provide the requisite cationic charge density.

A galactomannan polymer derivative can be a cationic derivative of the non-guar galactomannan polymer, which is obtained by reaction between the hydroxyl groups of the polygalactomannan polymer and reactive quaternary ammonium compounds. Suitable quaternary ammonium compounds for use in forming the cationic galactomannan polymer derivatives include those conforming to the general Formulas II to VI, as defined above.

Cationic non-guar galactomannan polymer derivatives formed from the reagents described above can be represented by the general Formula VII:

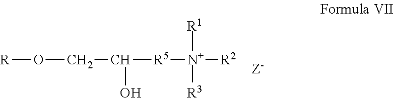

wherein R is the gum. The cationic galactomannan derivative can be a gum hydroxypropyltrimethylammonium chloride, which can be more specifically represented by the general Formula VIII:

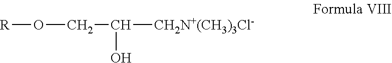

The galactomannan polymer derivative can be an amphoteric galactomannan polymer derivative having a net positive charge, obtained when the cationic galactomannan polymer derivative further comprises an anionic group.

A cationic non-guar galactomannan can have a ratio of mannose to galactose which is greater than 4:1, a M.Wt. of about 100,000 g/mol to about 500,000 g/mol, a M.Wt. of about 50,000 g/mol to about 400,000 g/mol, and a cationic charge density from about 1 meq/g to about 5 meq/g, and from about 2 meq/g to about 4 meq/g.

Cleansing compositions can include at least about 0.05% of a galactomannan polymer derivative by weight of the composition. The cleansing compositions can include from about 0.05% to about 2%, by weight of the composition, of a galactomannan polymer derivative.

### Cationic Starch Polymers

Suitable cationic polymers can also be water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cleansing compositions described herein can include cationically modified starch polymers at a range of about 0.01% to about 10%, and/or from about 0.05% to about 5%, by weight of the composition.

The cationically modified starch polymers disclosed herein have a percent of bound nitrogen of from about 0.5% to about 4%.

The cationically modified starch polymers can have a molecular weight from about 850,000 g/mol to about 15,000,000 g/mol and from about 900,000 g/mol to about 5,000,000 g/mol.

Cationically modified starch polymers can have a charge density of from about 0.2 meq/g to about 5 meq/g, and from about 0.2 meq/g to about 2 meq/g. The chemical modification to obtain such a charge density can include the addition of amino and/or ammonium groups into the starch molecules. Non-limiting examples of such ammonium groups can include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. Further details are described in Solarek, D. B., Cationic Starches in Modified Starches: Properties and Uses, Wurzburg, O. B., Ed., CRC Press, Inc., Boca Raton, Fla. 1986, pp 113-125 which is hereby incorporated by reference. The cationic groups can be added to the starch prior to degradation to a smaller molecular weight or the cationic groups may be added after such modification.

A cationically modified starch polymer can have a degree of substitution of a cationic group from about 0.2 to about 2.5. As used herein, the “degree of substitution” of the cationically modified starch polymers is an average measure of the number of hydroxyl groups on each anhydroglucose unit which is derivatized by substituent groups. Since each anhydroglucose unit has three potential hydroxyl groups available for substitution, the maximum possible degree of substitution is 3. The degree of substitution is expressed as the number of moles of substituent groups per mole of anhydroglucose unit, on a molar average basis. The degree of substitution can be determined using proton nuclear magnetic resonance spectroscopy (“¹H NMR”) methods well known in the art. Suitable 1H NMR techniques include those described in “Observation on NMR Spectra of Starches in Dimethyl Sulfoxide, Iodine-Complexing, and Solvating in Water-Dimethyl Sulfoxide”, Qin-Ji Peng and Arthur S. Perlin, Carbohydrate Research, 160 (1987), 57-72; and “An Approach to the Structural Analysis of Oligosaccharides by NMR Spectroscopy”, J. Howard Bradbury and J. Grant Collins, Carbohydrate Research, 71, (1979), 15-25.

The source of starch before chemical modification can be selected from a variety of sources such as tubers, legumes, cereal, and grains. For example, starch sources can include corn starch, wheat starch, rice starch, waxy corn starch, oat starch, cassaya starch, waxy barley, waxy rice starch, glutenous rice starch, sweet rice starch, amioca, potato starch, tapioca starch, oat starch, sago starch, sweet rice, or mixtures thereof. Suitable cationically modified starch polymers can be selected from degraded cationic maize starch, cationic tapioca, cationic potato starch, and mixtures thereof. Cationically modified starch polymers are cationic corn starch and cationic tapioca.

The starch, prior to degradation or after modification to a smaller molecular weight, can include one or more additional modifications. For example, these modifications may include cross-linking, stabilization reactions, phosphorylations, and hydrolyzations. Stabilization reactions can include alkylation and esterification.

Cationically modified starch polymers can be included in a cleansing composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

The starch can be readily soluble in water and can form a substantially translucent solution in water. The transparency of the composition is measured by Ultra-Violet/Visible (“UV/VIS”) spectrophotometry, which determines the absorption or transmission of UV/VIS light by a sample, using a Gretag Macbeth Colorimeter Color. A light wavelength of 600 nm has been shown to be adequate for characterizing the degree of clarity of cleansing compositions.

### Cationic Copolymer of an Acrylamide Monomer and a Cationic Monomer

A cleansing composition can include a cationic copolymer of an acrylamide monomer and a cationic monomer, wherein the copolymer has a charge density of from about 1.0 meq/g to about 3.0 meq/g. The cationic copolymer can be a synthetic cationic copolymer of acrylamide monomers and cationic monomers.

Suitable cationic polymers can include:
(i) an acrylamide monomer of the following Formula IX:

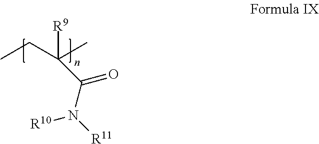

where R⁹ is H or C₁₋₄ alkyl; and R¹⁰ and R¹¹ are independently selected from the group consisting of H, C₁₋₄ alkyl, CH₂OCH₃, CH₂OCH₂CH(CH₃)₂, and phenyl, or together are C₃₋₆cycloalkyl; and (ii) a cationic monomer conforming to Formula X:

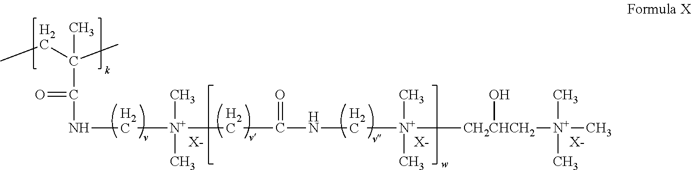

where k=1, each of v, v′, and v″ is independently an integer of from 1 to 6, w is zero or an integer of from 1 to 10, and X-is an anion.

A cationic monomer can conform to Formula X where k=1, v=3 and w=0, z=1 and X⁻ is Cl⁻ to form the following structure (Formula XI):

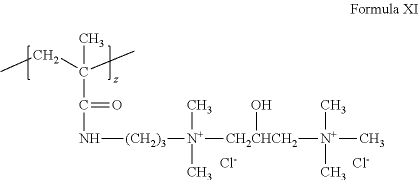

As can be appreciated, the above structure can be referred to as diquat.

A cationic monomer can conform to Formula X wherein v and v″ are each 3, v′=1, w=1, y=1 and X is Cl⁻, to form the following structure of Formula XII:

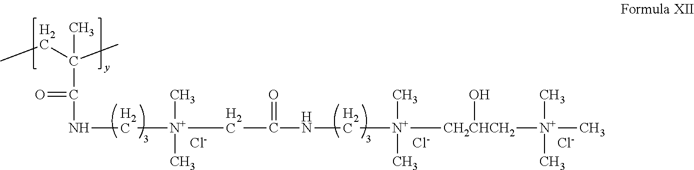

The structure of Formula XII can be referred to as triquat.

The acrylamide monomer can be either acrylamide or methacrylamide.

The cationic copolymer can be AM:TRIQUAT which is a copolymer of acrylamide and 1,3-Propanediaminium, N-[2-[[[dimethyl[3-[(2-methyl-1-oxo-2-propenyl)amino]propyl]ammonio]acetyl]amino]ethyl]2-hydroxy-N,N,N′,N′,N′-pentamethyl-, trichloride. AM:TRIQUAT is also known as polyquaternium 76 (PQ76). AM:TRIQUAT can have a charge density of 1.6 meq/g and a M.Wt. of 1.1 million g/mol.

The cationic copolymer can include an acrylamide monomer and a cationic monomer, wherein the cationic monomer is selected from the group consisting of: dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide; ethylenimine, vinylamine, 2-vinylpyridine, 4-vinylpyridine; trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride, and mixtures thereof.

The cationic copolymer can include a cationic monomer selected from the group consisting of: trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, and mixtures thereof.

The cationic copolymer can be formed from (1) copolymers of (meth)acrylamide and cationic monomers based on (meth)acrylamide, and/or hydrolysis-stable cationic monomers, (2) terpolymers of (meth)acrylamide, monomers based on cationic (meth)acrylic acid esters, and monomers based on (meth)acrylamide, and/or hydrolysis-stable cationic monomers. Monomers based on cationic (meth)acrylic acid esters can be cationized esters of the (meth)acrylic acid containing a quaternized N atom. Cationized esters of the (meth)acrylic acid containing a quaternized N atom can be quaternized dialkylaminoalkyl (meth)acrylates with C1 to C3 in the alkyl and alkylene groups. The cationized esters of the (meth)acrylic acid containing a quaternized N atom can be selected from the group consisting of: ammonium salts of dimethylaminomethyl (meth)acrylate, dimethylaminoethyl (meth)acrylate, dimethylaminopropyl (meth)acrylate, diethylaminomethyl (meth)acrylate, diethylaminoethyl (meth)acrylate; and diethylaminopropyl (meth)acrylate quaternized with methyl chloride. The cationized esters of the (meth)acrylic acid containing a quaternized N atom can be dimethylaminoethyl acrylate, which is quaternized with an alkyl halide, or with methyl chloride or benzyl chloride or dimethyl sulfate (ADAME-Quat). The cationic monomer when based on (meth)acrylamides are quaternized dialkylaminoalkyl (meth)acrylamides with C₁ to C₃ in the alkyl and alkylene groups, or dimethylaminopropylacrylamide, which is quaternized with an alkyl halide, or methyl chloride or benzyl chloride or dimethyl sulfate.

The cationic monomer based on a (meth)acrylamide can be a quaternized dialkylaminoalkyl (meth)acrylamide with C₁ to C₃ in the alkyl and alkylene groups. The cationic monomer based on a (meth)acrylamide can be dimethylaminopropylacrylamide, which is quaternized with an alkyl halide, especially methyl chloride or benzyl chloride or dimethyl sulfate. The cationic monomer can be a hydrolysis-stable cationic monomer. Hydrolysis-stable cationic monomers can be, in addition to a dialkylaminoalkyl (meth)acrylamide, any monomer that can be regarded as stable to the OECD hydrolysis test. The cationic monomer can be hydrolysis-stable, and the hydrolysis-stable cationic monomer can be selected from the group consisting of: diallyldimethylammonium chloride and water-soluble, cationic styrene derivatives.

The cationic copolymer can be a terpolymer of acrylamide, 2-dimethylammoniumethyl (meth)acrylate quaternized with methyl chloride (ADAME-Q) and 3-dimethylammoniumpropyl (meth)acrylamide quaternized with methyl chloride (DIMAPA-Q).

The cationic copolymer can be formed from acrylamide and acrylamidopropyltrimethylammonium chloride, wherein the acrylamidopropyltrimethylammonium chloride has a charge density of from about 1.0 meq/g to about 3.0 meq/g.

The cationic copolymer can have a charge density of from about 1.1 meq/g to about 2.5 meq/g, from about 1.1 meq/g to about 2.3 meq/g, from about 1.2 meq/g to about 2.2 meq/g, from about 1.2 meq/g to about 2.1 meq/g, from about 1.3 meq/g to about 2.0 meq/g, and from about 1.3 meq/g to about 1.9 meq/g.

The cationic copolymer can have a M.Wt. from about 100 thousand g/mol to about 2 million g/mol, from about 300 thousand g/mol to about 1.8 million g/mol, from about 500 thousand g/mol to about 1.6 million g/mol, from about 700 thousand g/mol to about 1.4 million g/mol, and from about 900 thousand g/mol to about 1.2 million g/mol.

The cationic copolymer can be a trimethylammoniopropylmethacrylamide chloride-N-Acrylamide copolymer, which is also known as AM:MAPTAC. AM:MAPTAC can have a charge density of about 1.3 meq/g and a M.Wt. of about 1.1 million g/mol. The cationic copolymer can be AM:ATPAC. AM:ATPAC can have a charge density of about 1.8 meq/g and a M.Wt. of about 1.1 million g/mol.

### Synthetic Polymers

A cationic polymer can be a synthetic polymer that is formed from:
i) one or more cationic monomer units, and optionally
ii) one or more monomer units bearing a negative charge, and/or
iii) a nonionic monomer,

wherein the subsequent charge of the copolymer is positive. The ratio of the three types of monomers is given by “m”, “p” and “q” where “m” is the number of cationic monomers, “p” is the number of monomers bearing a negative charge and “q” is the number of nonionic monomers

The cationic polymers can be water soluble or dispersible, non-crosslinked, and synthetic cationic polymers which have the structure of Formula XIII:

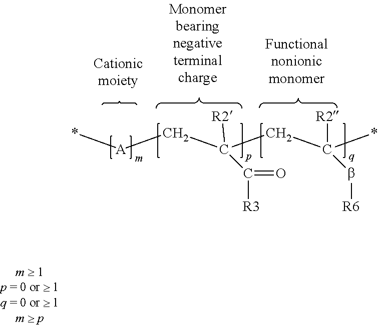

where A, may be one or more of the following cationic moieties:

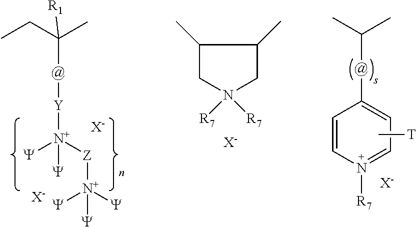

where @=amido, alkylamido, ester, ether, alkyl or alkylaryl;
where Y=C1-C22 alkyl, alkoxy, alkylidene, alkyl or aryloxy;
where ψ=C1-C22 alkyl, alkyloxy, alkyl aryl or alkyl arylox;.
where Z=C1-C22 alkyl, alkyloxy, aryl or aryloxy;
where R1=H, C1-C4 linear or branched alkyl;
where s=0 or 1, n=0 or ≥1;
where T and R7=C1-C22 alkyl; and
where X-=halogen, hydroxide, alkoxide, sulfate or alkylsulfate.

Where the monomer bearing a negative charge is defined by R2′=H, C₁-C₄ linear or branched alkyl and R3 is:

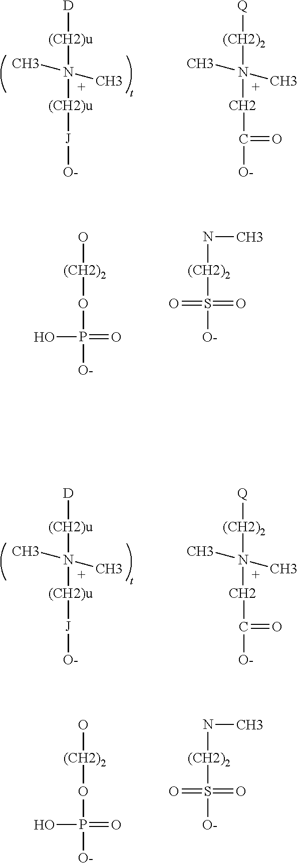

where D=O, N, or S;
where Q=NH2 or O;
where u=1-6;
where t=0-1; and
where J=oxygenated functional group containing the following elements P, S, C.
   Where the nonionic monomer is defined by R2″=H, C₁-C₄ linear or branched alkyl, R6=linear or branched alkyl, alkyl aryl, aryl oxy, alkyloxy, alkylaryl oxy and β is defined as

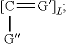

and
where G′ and G″ are, independently of one another, O, S or N—H and L=0 or 1.

Suitable monomers can include aminoalkyl (meth)acrylates, (meth)aminoalkyl (meth)acrylamides; monomers comprising at least one secondary, tertiary or quaternary amine function, or a heterocyclic group containing a nitrogen atom, vinylamine or ethylenimine; diallyldialkyl ammonium salts; their mixtures, their salts, and macromonomers deriving from therefrom.

Further examples of suitable cationic monomers can include dimethylaminoethyl (meth)acrylate, dimethylaminopropyl (meth)acrylate, ditertiobutylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine, trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride, diallyldimethyl ammonium chloride.

Suitable cationic monomers can include quaternary monomers of formula —NR₃⁺, wherein each R can be identical or different, and can be a hydrogen atom, an alkyl group comprising 1 to 10 carbon atoms, or a benzyl group, optionally carrying a hydroxyl group, and including an anion (counter-ion). Examples of suitable anions include halides such as chlorides, bromides, sulphates, hydrosulphates, alkylsulphates (for example comprising 1 to 6 carbon atoms), phosphates, citrates, formates, and acetates.

Suitable cationic monomers can also include trimethylammonium ethyl (meth)acrylate chloride, trimethylammonium ethyl (meth)acrylate methyl sulphate, dimethylammonium ethyl (meth)acrylate benzyl chloride, 4-benzoylbenzyl dimethylammonium ethyl acrylate chloride, trimethyl ammonium ethyl (meth)acrylamido chloride, trimethyl ammonium propyl (meth)acrylamido chloride, vinylbenzyl trimethyl ammonium chloride. Additional suitable cationic monomers can include trimethyl ammonium propyl (meth)acrylamido chloride.

Examples of monomers bearing a negative charge include alpha ethylenically unsaturated monomers including a phosphate or phosphonate group, alpha ethylenically unsaturated monocarboxylic acids, monoalkylesters of alpha ethylenically unsaturated dicarboxylic acids, monoalkylamides of alpha ethylenically unsaturated dicarboxylic acids, alpha ethylenically unsaturated compounds comprising a sulphonic acid group, and salts of alpha ethylenically unsaturated compounds comprising a sulphonic acid group.

Suitable monomers with a negative charge can include acrylic acid, methacrylic acid, vinyl sulphonic acid, salts of vinyl sulfonic acid, vinylbenzene sulphonic acid, salts of vinylbenzene sulphonic acid, alpha-acrylamidomethylpropanesulphonic acid, salts of alpha-acrylamidomethylpropanesulphonic acid, 2-sulphoethyl methacrylate, salts of 2-sulphoethyl methacrylate, acrylamido-2-methylpropanesulphonic acid (AMPS), salts of acrylamido-2-methylpropanesulphonic acid, and styrenesulphonate (SS).

Examples of nonionic monomers can include vinyl acetate, amides of alpha ethylenically unsaturated carboxylic acids, esters of an alpha ethylenically unsaturated monocarboxylic acids with an hydrogenated or fluorinated alcohol, polyethylene oxide (meth)acrylate (i.e. polyethoxylated (meth)acrylic acid), monoalkylesters of alpha ethylenically unsaturated dicarboxylic acids, monoalkylamides of alpha ethylenically unsaturated dicarboxylic acids, vinyl nitriles, vinylamine amides, vinyl alcohol, vinyl pyrolidone, and vinyl aromatic compounds. Suitable nonionic monomers can also include styrene, acrylamide, methacrylamide, acrylonitrile, methylacrylate, ethylacrylate, n-propylacrylate, n-butylacrylate, methylmethacrylate, ethylmethacrylate, n-propylmethacrylate, n-butylmethacrylate, 2-ethyl-hexyl acrylate, 2-ethyl-hexyl methacrylate, 2-hydroxyethylacrylate and 2-hydroxyethylmethacrylate.

The anionic counterion (X⁻) in association with the synthetic cationic polymers can be any known counterion so long as the polymers remain soluble or dispersible in water, in the cleansing composition, or in a coacervate phase of the cleansing composition, and so long as the counterions are physically and chemically compatible with the essential components of the cleansing composition or do not otherwise unduly impair product performance, stability or aesthetics. Non limiting examples of suitable counterions can include halides (e.g., chlorine, fluorine, bromine, iodine), sulfate, and methylsulfate.

The cationic polymer described herein can also aid in repairing damaged hair, particularly chemically treated hair by providing a surrogate hydrophobic F-layer. The microscopically thin F-layer provides natural weatherproofing, while helping to seal in moisture and prevent further damage. Chemical treatments damage the hair cuticle and strip away its protective F-layer. As the F-layer is stripped away, the hair becomes increasingly hydrophilic. It has been found that when lyotropic liquid crystals are applied to chemically treated hair, the hair becomes more hydrophobic and more virgin-like, in both look and feel. Without being limited to any theory, it is believed that the lyotropic liquid crystal complex creates a hydrophobic layer or film, which coats the hair fibers and protects the hair, much like the natural F-layer protects the hair. The hydrophobic layer can return the hair to a generally virgin-like, healthier state.

Lyotropic liquid crystals are formed by combining the synthetic cationic polymers described herein with the aforementioned anionic detersive surfactant component of the cleansing composition. The synthetic cationic polymer has a relatively high charge density. It should be noted that some synthetic polymers having a relatively high cationic charge density do not form lyotropic liquid crystals, primarily due to their abnormal linear charge densities. Such synthetic cationic polymers are described in PCT Patent App. No. WO 94/06403 which is incorporated by reference. The synthetic polymers described herein can be formulated in a stable cleansing composition that provides improved conditioning performance, with respect to damaged hair.

Cationic synthetic polymers that can form lyotropic liquid crystals have a cationic charge density of from about 2 meq/gm to about 7 meq/gm, and/or from about 3 meq/gm to about 7 meq/gm, and/or from about 4 meq/gm to about 7 meq/gm. The cationic charge density is about 6.2 meq/gm. The polymers also have a M. Wt. of from about 1,000 to about 5,000,000, and/or from about 10,000 to about 2,000,000, and/or from about 100,000 to about 2,000,000.

Cationic synthetic polymers that provide enhanced conditioning and deposition of benefit agents but do not necessarily form lytropic liquid crystals can have a cationic charge density of from about 0.7 meq/gm to about 7 meq/gm, and/or from about 0.8 meq/gm to about 5 meq/gm, and/or from about 1.0 meq/gm to about 3 meq/gm. The polymers also have a M.Wt. of from about 1,000 g/mol to about 5,000,000 g/mol, from about 10,000 g/mol to about 2,000,000 g/mol, and from about 100,000 g/mol to about 2,000,000 g/mol.

### Cationic Cellulose Polymer

Suitable cationic polymers can be cellulose polymers. Suitable cellulose polymers can include salts of hydroxyethyl cellulose reacted with trimethyl ammonium substituted epoxide, referred to in the industry (CTFA) as Polyquaternium 10 and available from Dwo/Amerchol Corp. (Edison, N.J., USA) in their Polymer LR, JR, and KG series of polymers. Other suitable types of cationic cellulose can include the polymeric quaternary ammonium salts of hydroxyethyl cellulose reacted with lauryl dimethyl ammonium-substituted epoxide referred to in the industry (CTFA) as Polyquaternium 24. These materials are available from Dow/Amerchol Corp. under the tradename Polymer LM-200. Other suitable types of cationic cellulose can include the polymeric quaternary ammonium salts of hydroxyethyl cellulose reacted with lauryl dimethyl ammonium-substituted epoxide and trimethyl ammonium substituted epoxide referred to in the industry (CTFA) as Polyquaternium 67. These materials are available from Dow/Amerchol Corp. under the tradename SoftCAT Polymer SL-5, SoftCAT Polymer SL-30, Polymer SL-60, Polymer SL-100, Polymer SK-L, Polymer SK-M, Polymer SK-MH, and Polymer SK-H. Cationic cellulose polymers can have a cationic charge density of from about 0.2 meq/g to about 2.2 meq/g, from about 0.3 meq/g to about 2.0 meq/g, from about 0.4 meq/g to about 1.8 meq/g; from about 0.5 meq/g to about 1.7 meq/g and from about 0.6 meq/g to about 1.3.

Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

Techniques for analysis of formation of complex coacervates are known in the art. For example, microscopic analyses of the compositions, at any chosen stage of dilution, can be utilized to identify whether a coacervate phase has formed. Such coacervate phase can be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164 which is incorporated by reference.

### Liquid Carrier

As can be appreciated, cleansing compositions can desirably be in the form of pourable liquid under ambient conditions. Inclusion of an appropriate quantity of a liquid carrier can facilitate the formation of a cleansing composition having an appropriate viscosity and rheology. A cleansing composition can include, by weight of the composition, about 20% to about 95%, by weight, of a liquid carrier, and about 60% to about 85%, by weight, of a liquid carrier. The liquid carrier can be an aqueous carrier such as water.

### Optional Components

As can be appreciated, cleansing compositions described herein can include a variety of optional components to tailor the properties and characteristics of the composition. As can be appreciated, suitable optional components are well known and can generally include any components which are physically and chemically compatible with the essential components of the cleansing compositions described herein. Optional components should not otherwise unduly impair product stability, aesthetics, or performance. Individual concentrations of optional components can generally range from about 0.001% to about 10%, by weight of a cleansing composition. Optional components can be further limited to components which will not impair the clarity of a translucent cleansing composition.

Suitable optional components which can be included in a cleansing composition can include co-surfactants, deposition aids, conditioning agents (including hydrocarbon oils, fatty esters, silicones), anti-dandruff agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, skin active agents, sunscreens, UV absorbers, and vitamins. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of non-limiting materials that can be added to the composition herein.

### Conditioning Agents

A cleansing composition can include a silicone conditioning agent. Suitable silicone conditioning agents can include volatile silicone, non-volatile silicone, or combinations thereof. If including a silicone conditioning agent, the agent can be included from about 0.01% to about 10%, by weight of the composition, from about 0.1% to about 8%, from about 0.1% to about 5%, and/or from about 0.2% to about 3%. Examples of suitable silicone conditioning agents, and optional suspending agents for the silicone, are described in U.S. Reissue Pat. No. 34,584, U.S. Pat. Nos. 5,104,646, and 5,106,609, each of which is incorporated by reference herein. Suitable silicone conditioning agents can have a viscosity, as measured at 25° C., from about 20 centistokes (“csk”) to about 2,000,000 csk, from about 1,000 csk to about 1,800,000 csk, from about 50,000 csk to about 1,500,000 csk, and from about 100,000 csk to about 1,500,000 csk.

The dispersed silicone conditioning agent particles can have a volume average particle diameter ranging from about 0.01 micrometer to about 50 micrometer. For small particle application to hair, the volume average particle diameters can range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, and/or from about 20 micrometer to about 50 micrometer.

Additional material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), which is incorporated herein by reference.

Silicone emulsions suitable for the cleansing compositions described herein can include emulsions of insoluble polysiloxanes prepared in accordance with the descriptions provided in U.S. Pat. No. 4,476,282 and U.S. Patent Application Publication No. 2007/0276087 each of which is incorporated herein by reference. Suitable insoluble polysiloxanes include polysiloxanes such as alpha, omega hydroxy-terminated polysiloxanes or alpha, omega alkoxy-terminated polysiloxanes having a molecular weight within the range from about 50,000 to about 500,000 g/mol. The insoluble polysiloxane can have an average molecular weight within the range from about 50,000 to about 500,000 g/mol. For example, the insoluble polysiloxane may have an average molecular weight within the range from about 60,000 to about 400,000; from about 75,000 to about 300,000; from about 100,000 to about 200,000; or the average molecular weight may be about 150,000 g/mol. The insoluble polysiloxane can have an average particle size within the range from about 30 nm to about 10 micron. The average particle size may be within the range from about 40 nm to about 5 micron, from about 50 nm to about 1 micron, from about 75 nm to about 500 nm, or about 100 nm, for example.

Other classes of silicones suitable for the cleansing compositions described herein can include i) silicone fluids, including silicone oils, which are flowable materials having viscosity less than about 1,000,000 csk as measured at 25° C.; ii) aminosilicones, which contain at least one primary, secondary or tertiary amine; iii) cationic silicones, which contain at least one quaternary ammonium functional group; iv) silicone gums; which include materials having viscosity greater or equal to 1,000,000 csk as measured at 25° C.; v) silicone resins, which include highly cross-linked polymeric siloxane systems; vi) high refractive index silicones, having refractive index of at least 1.46, and vii) mixtures thereof.

Alternatively, the cleansing composition can be substantially free or free of silicones.

### Organic Conditioning Materials

The conditioning agent of the cleansing compositions described herein can also include at least one organic conditioning material such as oil or wax, either alone or in combination with other conditioning agents, such as the silicones described above. The organic material can be non-polymeric, oligomeric or polymeric. The organic material can be in the form of an oil or wax and can be added in the cleansing formulation neat or in a pre-emulsified form. Suitable examples of organic conditioning materials can include: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-200, PEG-400, PEG-600, PEG-1000, PEG-2M, PEG-7M, PEG-14M, PEG-45M and mixtures thereof.

### Emulsifiers

A variety of anionic and nonionic emulsifiers can be used in the cleansing composition of the present invention. The anionic and nonionic emulsifiers can be either monomeric or polymeric in nature. Monomeric examples include, by way of illustrating and not limitation, alkyl ethoxylates, alkyl sulfates, soaps, and fatty esters and their derivatives. Polymeric examples include, by way of illustrating and not limitation, polyacrylates, polyethylene glycols, and block copolymers and their derivatives. Naturally occurring emulsifiers such as lanolins, lecithin and lignin and their derivatives are also non-limiting examples of useful emulsifiers.

### Chelating Agents

The cleansing composition can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term includes alkali metal, alkaline earth, ammonium, substituted ammonium (i.e. monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. Nos. 5,747,440. 5,284,972 and 5,747,440 are each incorporated by reference herein. Suitable chelants can further include histidine.

Levels of an EDDS chelant or histidine chelant in the cleansing compositions can be low. For example, an EDDS chelant or histidine chelant can be included at about 0.01%, by weight. Above about 10% by weight, formulation and/or human safety concerns can arise. The level of an EDDS chelant or histidine chelant can be at least about 0.01%, by weight, at least about 0.05%, by weight, at least about 0.1%, by weight, at least about 0.25%, by weight, at least about 0.5%, by weight, at least about 1%, by weight, or at least about 2%, by weight, by weight of the cleansing composition.

### Gel Network

A cleansing composition can also include a fatty alcohol gel network. Gel networks are formed by combining fatty alcohols and surfactants in the ratio of from about 1:1 to about 40:1, from about 2:1 to about 20:1, and/or from about 3:1 to about 10:1. The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. Gel networks can provide a number of benefits to cleansing compositions. For example, a gel network can provide a stabilizing benefit to cosmetic creams and hair conditioners. In addition, gel networks can provide conditioned feel benefits to hair conditioners and shampoos.

A fatty alcohol can be included in the gel network at a level by weight of from about 0.05%, by weight, to about 14%, by weight. For example, the fatty alcohol can be included in an amount ranging from about 1%, by weight, to about 10%, by weight,, and/or from about 6%, by weight, to about 8%, by weight.

Suitable fatty alcohols include those having from about 10 to about 40 carbon atoms, from about 12 to about 22 carbon atoms, from about 16 to about 22 carbon atoms, and/or about 16 to about 18 carbon atoms. These fatty alcohols can be straight or branched chain alcohols and can be saturated or unsaturated. Nonlimiting examples of fatty alcohols include cetyl alcohol, stearyl alcohol, behenyl alcohol, and mixtures thereof. Mixtures of cetyl and stearyl alcohol in a ratio of from about 20:80 to about 80:20 are suitable.

A gel network can be prepared by charging a vessel with water. The water can then be heated to about 74° C. Cetyl alcohol, stearyl alcohol, and surfactant can then be added to the heated water. After incorporation, the resulting mixture can pass through a heat exchanger where the mixture is cooled to about 35° C. Upon cooling, the fatty alcohols and surfactant crystallized can form crystalline gel network. Table 1 provides the components and their respective amounts for an example gel network composition.

To prepare the gel network pre-mix of Table 1, water is heated to about 74° C. and the fatty alcohol and gel network surfactant are added to it in the quantities depicted in Table 1. After incorporation, this mixture is passed through a mill and heat exchanger where it is cooled to about 32° C. As a result of this cooling step, the fatty alcohol, the gel network surfactant, and the water form a crystalline gel network.

TABLE 1

| Premix | % |
|---|---|
| Gel Network Surfactant¹ | 11.00 |
| Stearyl Alcohol | 8% |
| Cetyl Alcohol | 4% |
| Water | QS |

¹For anionic gel networks, suitable gel network surfactants above include surfactants with a net negative charge including sulfonates, carboxylates and phosphates among others and mixtures thereof.

For cationic gel networks, suitable gel network surfactants above include surfactants with a net positive charge including quaternary ammonium surfactants and mixtures thereof.

For Amphoteric or Zwitterionic gel networks, suitable gel network surfactants above include surfactants with both a positive and negative charge at product usage pH including betaines, amine oxides, sultaines, amino acids among others and mixtures thereof.

### Benefit Agents

A cleansing composition can further include one or more benefit agents. Exemplary benefit agents include, but are not limited to, particles, colorants, perfume microcapsules, gel networks, and other insoluble skin or hair conditioning agents such as skin silicones, natural oils such as sunflower oil or castor oil. The benefit agent can be selected from the group consisting of particles; colorants; perfume microcapsules; gel networks; other insoluble skin or hair conditioning agents such as skin silicones, natural oils such as sunflower oil or castor oil; and mixtures thereof.

### Suspending Agent

A cleansing composition can include a suspending agent at concentrations effective for suspending water-insoluble material in dispersed form in the compositions or for modifying the viscosity of the composition. Such concentrations range from about 0.1% to about 10%, and from about 0.3% to about 5.0%, by weight of the compositions. As can be appreciated however, suspending agents may not be necessary when certain glyceride ester crystals are included as certain glyceride ester crystals can act as suitable suspending or structuring agents.

Suitable suspending agents can include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Other suitable suspending agents can include crystalline suspending agents which can be categorized as acyl derivatives, long chain amine oxides, and mixtures thereof. Examples of such suspending agents are described in U.S. Pat. No. 4,741,855, which is incorporated herein by reference. Suitable suspending agents include ethylene glycol esters of fatty acids having from 16 to 22 carbon atoms. The suspending agent can be an ethylene glycol stearates, both mono and distearate, but particularly the distearate containing less than about 7% of the mono stearate. Other suitable suspending agents include alkanol amides of fatty acids, having from about 16 to about 22 carbon atoms, alternatively from about 16 to about 18 carbon atoms, suitable examples include stearic stearic of which monoethanolamide, diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters as previously described. Long chain acyl derivatives, ethylene glycol esters of long chain carboxylic acids, long chain amine oxides, and alkanol amides of long chain carboxylic acids can also be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C₁₆, C₁₈ and tallow amido benzoic acid species of this family, which are commercially available from Stepan® Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

### Viscosity Modifiers

The shampoo composition can be free of or substantially free of viscosity modifiers other than organic salt.

In some examples, the composition can contain a viscosity modifier instead of or in addition to organic salt. Viscosity modifiers can be used to modify the rheology of a cleansing composition. Suitable viscosity modifiers can include Carbomers with tradenames Carbopol 934, Carbopol 940, Carbopol 950, Carbopol 980, and Carbopol 981, all available from B. F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied by Amerchol. Other suitable rheology modifiers can include cross-linked acrylates, cross-linked maleic anhydride co-methylvinylethers, hydrophobically modified associative polymers, and mixtures thereof.

### Dispersed Particles

Dispersed particles as known in the art can be included in a cleansing composition. If including such dispersed particles, the particles can be incorporated, by weight of the composition, at levels of about 0.025% or more, about 0.05% or more, about 0.1% or more, about 0.25% or more, and about 0.5% or more. However, the cleansing compositions can also contain, by weight of the composition, about 20% or fewer dispersed particles, about 10% or fewer dispersed particles, about 5% or fewer dispersed particles, about 3% or fewer dispersed particles, and about 2% or fewer dispersed particles.

As can be appreciated, a cleansing composition can include still further optional components. For example, amino acids can be included. Suitable amino acids can include water soluble vitamins such as vitamins B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

Anti-dandruff agents can be included. As can be appreciated, the formation of a coacervate can facilitate deposition of the anti-dandruff agent to the scalp.

Suitable anti-dandruff agents can include pyridinethione salts, azoles, selenium sulfide, particulate sulfur, and mixtures thereof. Such anti-dandruff particulate should be physically and chemically compatible with the essential components of the composition, and should not otherwise unduly impair product stability, aesthetics or performance. A shampoo composition can include a cationic polymer to enhance deposition of an anti-dandruff active.

An anti-dandruff agent can be a pyridinethione particulate such as a 1-hydroxy-2-pyridinethione salt. The concentration of pyridinethione anti-dandruff particulates can range from about 0.1% to about 4%, about 0.1% to about 3%, and from about 0.3% to about 2% by weight of the composition. Suitable pyridinethione salts include those formed from heavy metals such as zinc, tin, cadmium, magnesium, aluminum and zirconium, particularly suitable is the zinc salt of 1-hydroxy-2-pyridinethione (known as “zinc pyridinethione” or “ZPT”), 1-hydroxy-2-pyridinethione salts in platelet particle form, wherein the particles have an average size of up to about 20 μm, up to about 5 μm, up to about 2.5 μm. Salts formed from other cations, such as sodium, can also be suitable. Pyridinethione anti-dandruff agents are further described in U.S. Pat. Nos. 2,809,971; 3,236,733; 3,753,196; 3,761,418; 4,345,080; 4,323,683; 4,379,753; and 4,470,982, each of which are incorporated herein by reference. It is contemplated that when ZPT is used as the anti-dandruff particulate, that the growth or re-growth of hair may be stimulated or regulated, or both, or that hair loss may be reduced or inhibited, or that hair may appear thicker or fuller.

In addition to the anti-dandruff active selected from polyvalent metal salts of pyrithione, a shampoo composition can further include one or more anti-fungal or anti-microbial actives in addition to the metal pyrithione salt actives. Suitable anti-microbial actives include coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Suitable anti-microbials can include itraconazole, ketoconazole, selenium sulphide and coal tar.

A suitable anti-microbial agent can be one material or a mixture selected from: azoles, such as climbazole, ketoconazole, itraconazole, econazole, and elubiol; hydroxy pyridones, such as piroctone olamine, ciclopirox, rilopirox, and MEA-Hydroxyoctyloxypyridinone; kerolytic agents, such as salicylic acid and other hydroxy acids; strobilurins such as azoxystrobin and metal chelators such as 1,10-phenanthroline. Examples of azole anti-microbials can include imidazoles such as benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and triazoles such as terconazole and itraconazole, and combinations thereof. When present in a shampoo composition, the soluble anti-microbial active can be included in an amount from about 0.01% to about 5%, from about 0.5% to about 6%, from about 0.1% to about 3%, from about 0.1% to about 9%, from about 0.1% to about 1.5%, from about 0.1% to about 2%, and more from about 0.3% to about 2%, by weight of the composition.

Selenium sulfide is a particulate anti-dandruff agent suitable for use as an anti-microbial compositions when included at concentrations of about 0.1% to about 4%, by weight of the composition, from about 0.3% to about 2.5% by weight, and from about 0.5% to about 1.5% by weight. Selenium sulfide is generally regarded as a compound having one mole of selenium and two moles of sulfur, although it may also be a cyclic structure that conforms to the general formula SeₓS_{y}, wherein x+y=8. Average particle diameters for the selenium sulfide are typically less than 15 [symbol] m, as measured by forward laser light scattering device (e.g. Malvern 3600 instrument), less than 10 [symbol] m. Selenium sulfide compounds are described, for example, in U.S. Pat. Nos. 2,694,668; 3,152,046; 4,089,945; and 4,885,107, each of which are incorporated herein by reference.

Sulfur can also be used as a particulate anti-microbial/anti-dandruff agent. Effective concentrations of the particulate sulfur are typically from about 1% to about 4%, by weight of the composition, alternatively from about 2% to about 4%.

Keratolytic agents such as salicylic acid can also be included in a shampoo composition described herein.

A cleansing composition can optionally include pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including water soluble components such as those having C. I. Names.

One or more stabilizers and preservatives can be included. For example, one or more of trihydroxystearin, ethylene glycol distearate, citric acid, sodium citrate dihydrate, a preservative such as kathon, sodium chloride, sodium benzoate, sodium salicylate and ethylenediaminetetraacetic acid (“EDTA”) can be included to improve the lifespan of a personal care compositon. The stabilizer and/or preservative can be used at a level of from about 0.10% to about 2%. Particularly suitable is sodium benzoate at a level of from about 0.10% to about 0.45%. The personal care composition may also include citric acid at a level of from about 0.5% to about 2%. The sodium benzoate and the citric acid can be added to the personal care composition alone or in combination.

### Method of Making a Cleansing Composition

A cleansing composition described herein can be formed similarly to known cleansing compositions. For example, the process of making a cleansing composition can include the step of mixing the surfactant, cationic polymer, and liquid carrier together to form a cleansing composition. The sclerotium gum can be incorporated into the composition by dispersing it first in water, at about a 1:80 polymer-to-water ratio respectively, using a high shear milling device (e.g. IKA T25 DS1 Digital Ultra Turrax homogenizer), adjusting the mill speed and mill time to achieve the desired batch viscosity and rheology. The remaining ingredients were then added to complete the batch.

Additional information on sulfate-free surfactants and other ingredients that are suitable for shampoo compositions is found at U.S. Pub. Nos. 2019/0105247 and 2019/0105246, incorporated by reference.

### TEST METHODS

### Argentometry Method to Measure wt % Inorganic Chloride Salts

The weight % of inorganic chloride salt in the composition can be measured using a potentiometric method where the chloride ions in the composition are titrated with silver nitrate. The silver ions react with the chloride ions from the composition to form an insoluble precipitate, silver chloride. The method used an electrode (Mettler Toledeo DM141) that is designed for potentiometric titrations of anions that precipitate with silver. The largest change in the signal occurs at the equivalence point when the amount of added silver ions is equal to the amount of chloride ions in solution. The concentration of silver nitrate solution used should be calibrated using a chloride solution known to one of skill in the art, such as a sodium chloride solution that contains a standard and known amount of sodium chloride to confirm that the results match the known concentration. This type of titration involving a silver ion is known as argentometry and is commonly used to determine the amount of chloride present in a sample.

### Methods to Determine Lack of In Situ Coacervate in Composition Prior to Dilution

### 1. Microscopy Method to Determine Lack of In Situ Coacervate

Lack of in situ coacervate can be determined using a microscope. The composition is mixed to homogenize, if needed. Then, the composition is sampled onto a microscope slide and mounted on a microscope, per typical microscopy practices. The sample is viewed at, for example, a 10× or 20× objective. If in situ coacervate is present in the sample, an amorphous, gel-like phase with about 20 nm to about 200 nm particle size can be seen throughout the sample. This amorphous, gel-like phases can be described as gel chunks or globs. In this method, the in situ coacervate is separate from other ingredients that were intentionally added to the formula that form flocks or otherwise appear as particles under microscopy.

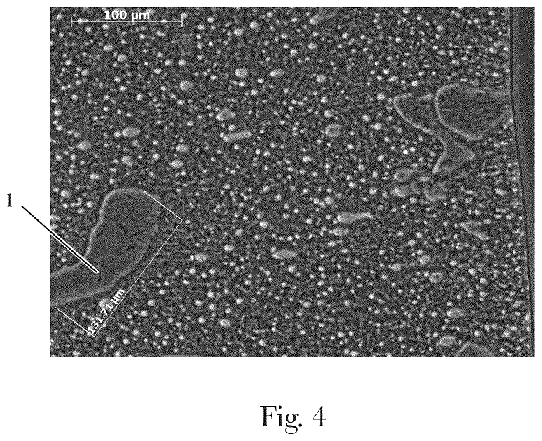

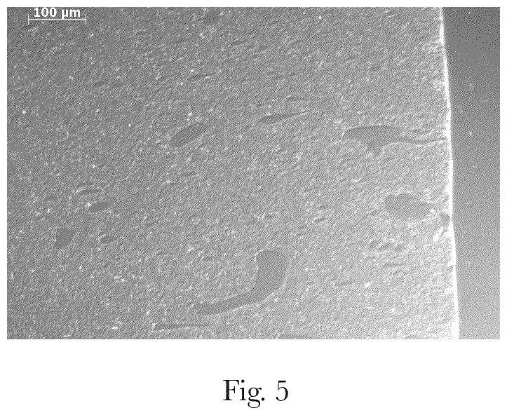

FIG. 4 is an example microscopy photograph at 20× objective of a marketed sulfate-free shampoo composition that contains a cationic polymer and also has in situ coacervate. FIG. 4 at reference numeral 1 shows an amorphous, gel-like phase that is about 130 nm long that is the in situ coacervate. FIG. 5 is an example microscopy photograph at 10× objective the same marketed shampoo composition that was used in FIG. 4 at 20× objective. FIG. 5 shows many of these amorphous, gel-like phases present with a length from about 20 nm to about 200 nm.

### 2. Clarity Assessment-Measurement of % Transmittance (% T)

Lack of in situ coacervate can be determined by composition clarity. A composition that does not contain in situ coacervate will be clear, if it does not contain any ingredients that would otherwise give it a hazy appearance.

Composition clarity can be measured by % Transmittance. For this assessment to determine if the composition lacks coacervate, the composition should be made without ingredients that would give the composition a hazy appearance such as silicones, opacifiers, non-silicone oils, micas, and gums or anionic rheology modifiers. It is believed that adding these ingredients would not cause in situ coacervate to form prior to use, however these ingredients will obscure measurement of clarity by % Transmittance.

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple samples are transferred into a 96 well visible flat bottom plate (Greiner part #655-001), ensuring that no air bubbles are within the samples. The flat bottom plate is placed within the SpectraMax M-5 and % T measured using the Software Pro v.5TM software available from Molecular Devices.

### 3. Lasentec FBRM Method

Lack of in situ coacervate can also be measured using Lasentec FBRM Method with no dilution. A Lasentec Focused Beam Reflectance Method (FBRM) [model S400A available from Mettler Toledo Corp] may be used to determine floc size and amount as measured by chord length and particle counts/sec (counts per sec).

### 4. In Situ Coacervate Centrifuge Method

Lack of in situ coacervate can also be measured by centrifuging a composition and measuring in situ coacervate gravimetrically. For this method, the composition should be made without a suspending agent to allow for separation of an in situ coacervate phase. The composition is centrifuged for 20 minutes at 9200 rpm using a Beckman Couller TJ25 centrifuge. Several time/rpm combinations can be used. The supernatant is then removed and the remaining settled in situ coacervate assessed gravimetrically. % In Situ Coacervate is calculated as the weight of settled in situ coacervate as a percentage of the weight of composition added to the centrifuge tube using the equation below. This quantifies the percentage of the composition that participates in the in situ coacervate phase.

$$\% In Situ Coacervate = \frac{Weight of settled in situ coacervate}{Weight of composition added to centrifuge tube} \times 100$$

### Measures of Improved Performance Due to no In Situ Coacervate Prior to Dilution

The composition does not contain in situ coacervate prior to dilution. Because of this, coacervate quantity and quality upon dilution is better than a composition that does contain in situ coacervate prior to dilution. This provides better wet conditioning and deposition of actives from a composition that does not contain coacervate prior to dilution compared to a composition that does contain coacervate prior to dilution.

### 1. Measurement of % Transmittance (% T) During Dilution

Coacervate formation upon dilution for a transparent or translucent composition can be assessed using a spectrophotometer to measure the percentage of light transmitted through the diluted sample (% T). As percent light transmittance (% T) values measured of the dilution decrease, typically higher levels of coacervate are formed. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1. By averaging the % T values for samples that span a range of dilution ratios, it is possible to simulate and ascertain how much coacervate a composition on average would form as a consumer applies the composition to wet hair, lathers, and then rinses it out. Average % T can be calculated by taking the numerical average of individual % T measurements for the following dilution ratios: 2:1, 3:1, 5:1, 7.5:1, 11:1, 16:1, 24:1, and 34:1. Lower average % T indicates more coacervate is formed on average as a consumer applies the composition to wet hair, lathers and then rinses it out.

% T can be measured using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to insure no air bubbles are within the sample before the % T is measured by the spectrophotometer at 600 nm. Alternatively, multiple samples can be measured simultaneously by using a spectrophotometer such as the SpectraMax M-5 available from Molecular Devices. Multiple dilution samples can be prepared within a 96 well plate (VWR catalog #82006-448) and then transferred to a 96 well visible flat bottom plate (Greiner part #655-001), ensuring that no air bubbles are within the sample. The flat bottom plate is placed within the SpectraMax M-5 and % T measured using the Software Pro v.5TM software available from Molecular Devices.

### 2. Assessment of Coacervate Floc Size Upon Dilution

Coacervate floc size upon dilution can be assessed visually. Dilutions samples at various weight ratios of water to composition can be prepared, for example 2 parts of water to 1 part composition (2:1), or 7.5 parts of water to 1 part composition (7.5:1), or 16 parts of water to 1 part composition (16:1), or 34 parts of water to 1 part composition (34:1), and the % T measured for each dilution ratio sample. Examples of possible dilution ratios may include 2:1, 3:1, 5:1, 7.5:1, 11:1, 16:1, 24:1, or 34:1.

### 3. Wet Combing Force Method

Hair switches of 4 grams general population hair at 8 inches length are used for the measurement. Each hair switch is treated with 4 cycles (1 lather/rinse steps per cycle, 0.1 gm cleansing composition/gm hair on each lather/rinse step, drying between each cycle) with the cleansing composition. Four switches are treated with each shampoo. The hair is not dried after the last treatment cycle. While the hair is wet, the hair is pulled through the fine tooth half of two Beautician 3000 combs. Force to pull the hair switch through the combs is measured by a friction analyzer (such as Instron or MTS tensile measurement) with a load cell and outputted in gram-force (gf). The pull is repeated for a total of five pulls per switch. Average wet combing force is calculated by averaging the force measurement from the five pulls across the four hair switches treated with each cleansing composition. Data can be shown as average wet combing force through one or both of the two combs.

### 4. Deposition Method

Deposition of actives can be measured in vitro on hair tresses or in vivo on panelist's heads. The composition is dosed on a hair tress or panelist head at a controlled amount and washed according to a conventional washing protocol. For a hair tress, the tress can be sampled and tested by an appropriate analytical measure to determine quantity deposited of a given active. To measure deposition on a panelist's scalp, the hair is then parted on an area of the scalp to allow an open-ended glass cylinder to be held on the surface while an aliquot of an extraction solution is added and agitated prior to recovery and analytical determination of a given active. To measure deposition on a panelist's hair, a given amount of hair is sampled and then tested by an appropriate analytical measure to determine quantity deposited of a given active.

### Cone/Plate Viscosity Measurement

The viscosities of the examples are measured by a Cone/Plate Controlled Stress Brookfield Rheometer R/S Plus, by Brookfield Engineering Laboratories, Stoughton, MA. The cone used (Spindle C-75-1) has a diameter of 75 mm and 1° angle. The liquid viscosity is determined using a steady state flow experiment at constant shear rate of 2 s⁻and at temperature of 26.7° C. The sample size is about 2.5 ml to about 3 ml and the total measurement reading time is 3 minutes.

### Lather Characterization-Kruss DFA100 Lather Characterization

A cleansing composition dilution of 10 parts by weight water to 1 part by weight cleanser is prepared. The shampoo dilution is dispensed into the Kruss DFA100 which generates the lather and measures lather properties.

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe. Submerse the electrode into the testing sample and press the read button. Wait until the pH icon stops flashing and record the value.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

The following Examples illustrate various shampoo compositions. The Examples in Table 2 and Table 3 were prepared by conventional formulation and mixing techniques. The sclerotium gum was incorporated using high shear milling, as described herein.

The total sodium chloride in the examples in Table 2 and Table 3 was 0.07%. The total sodium chloride in the tables below was calculated based on the product specifications from the suppliers. The ratio of anionic to amphoteric surfactant in Table 2 and Table 3 was 0.9:1. The ratio of anionic surfactant to amphoteric surfactant is calculated by wt. %. The ratio of polymer charge density to inorganic salt in Table 2 and Table 3 was 18:1.

The ratio of polymer charge density to inorganic salt is the charge density of the polymer (meq/gm) to the wt. % of the inorganic salt, disregarding the units. If the composition contains more than one cationic polymer, then the ratio is calculated according to the polymer with the lowest charge density.

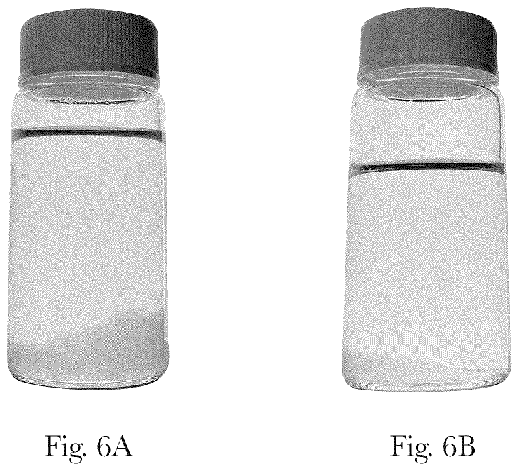

For the examples and comparative examples Table 2 and Table 3, the phase stability was determined as follows. The examples were prepared and immediately put in a clear, glass jar of at least 1 inch width. The cap was screwed on the jar, finger tight. The example was stored at ambient temperatures (20-25° C.), away from direct sunlight, for 5 days. Then the composition was inspected to see if either haze and/or precipitate was visually detectable. The haze and/or precipitate can be suspended across the liquid shampoo composition or a portion thereof and/or at or near the bottom of the container. If either haze or precipitate were present, it was determined that the composition had multiple phases and was not phases stable. FIGS. 6A and 6B show Comparative Example C5 (containing 0.5% konjac gum thickener) and C6 (containing 0.5% gellan gum thickener), respectively. A precipitate can be seen towards the bottom of the container in both FIGS. 6A and 6B. If neither haze nor precipitate were present, it was determined that there was no in situ coacervate and the composition was a stable, single phase. It is believed that the stable shampoo examples would have improved product performance as compared to the examples that were not phase stable.

As used herein, “visually detect” or “visually detectable” means that a human viewer can visually discern the quality of the example with the unaided eye (excepting standard corrective lenses adapted to compensate for near-sightedness, farsightedness, or stigmatism, or other corrected vision) in lighting at least equal to the illumination of a standard 100 watt incandescent white light bulb at a distance of 1 meter.

The examples in Table 2 and Table 3, could also be formulated with silicones, opacifiers (e.g. glycol distearate, glycol stearate), non-silicone oils, micas, gums or anionic rheology modifiers and other ingredients that would cause the shampoo to have a hazy or hazier appearance. However, it is believed that adding these ingredients would not cause in situ coacervate to form or other phase instability prior to use.

TABLE 2
Comparative Shampoo Composition Examples

| | C1 | C2 | C3 | C4 | C5 | C6 |
|---|---|---|---|---|---|---|
| | One | One | Multiple | Multiple | Multiple | Multiple |
| Phase Stability | phase | phase | phases | phases | phases | phases |
| pH | 5.62 | 5.54 | 5.75 | 5.81 | 5.79 | 5.87 |
| Viscosity @ 2 s-1 (cps) | 2236 | 893 | 4846 | 8588 | 3896 | 3053 |
| Lauramidopropyl Betaine ¹ | 9.75 | 9.75 | 9.75 | 9.75 | 9.75 | 9.75 |
| Sodium Cocoyl Isethionate ² | 6.00 | 6.00 | 6.00 | 6.00 | 6.00 | 6.00 |
| Sodium Lauryl Sarcosinate ³ | 2.50 | 2.50 | 2.50 | 2.50 | 2.50 | 2.50 |
| Polyquaternium 10 ⁴ | 0.55 | 0.25 | | 0.55 | 0.55 | 0.55 |
| (JR-30M, CD 1.25 meq/g) | | | | | | |
| Xanthan Gum ⁸ | | | 0.20 | 0.20 | | |
| Carrageenan ⁹ | | | 0.20 | 0.20 | | |
| Konjac Gum ¹⁰ | | | | | 0.50 | |
| Gellan Gum ¹¹ | | | | | | 0.50 |

| | |
|---|---|
| Citric Acid ¹² | To pH 5.5-6.1 |
| Water, Preservatives, | Q.S. to 100 |
| Perfume, and Optional | |
| Components | |

C1 and C2 in Table 2 were phase stable. However, neither example contains a thickener, and the viscosity is too low to be consumer preferred. It is suspected that these examples will be difficult to hold in a user's palm and apply across the hair.

C3-C6 contained an anionic thickener (e.g. xanthan gum, carrageenan, konjac gum, and gellan gum) separated into multiple phases, which is believed to not only impact the appearance of the product, but will also impact product performance. Thus, C3-C6 will likely not be consumer preferred.

Yield stress was not measured for the examples in Table 2 because the examples were not consumer acceptable.

TABLE 3
Shampoo Composition Examples

| | Ex. 1 | Ex. 2 | Ex. 3 | Ex. 4 |
|---|---|---|---|---|
| Phase Stability | One | One | One | One |
| | phase | phase | phase | phase |
| pH | 5.86 | 5.84 | 5.82 | 5.50 |
| Viscosity @ 2 s−1 (cps) | 4192 | 2681 | 4889 | 10615 |
| Yield Stress, Herschel- | 0.004 | 0.008 | 0.017 | 0.16 |
| Bulkley @ shear rate 10⁻² | | | | |
| to 10⁻⁴ s⁻¹ (Pa) | | | | |
| Lauramidopropyl Betaine ¹ | 9.75 | 9.75 | 9.75 | 9.75 |
| Sodium Cocoyl Isethionate ² | 6.00 | 6.00 | 6.00 | 6.00 |
| Sodium Lauryl Sarcosinate ³ | 2.50 | 2.50 | 2.50 | 2.50 |
| Polyquaternium 10 ⁴ | 0.55 | 0.55 | 0.55 | 0.55 |
| (JR-30M, CD 1.25 meq/g) | | | | |
| Sclerotium Gum ^{5,6} | 0.50 | 1.00 | | |
| Sclerotium Gum and Citrus | | | 0.50 | 1.00 |
| Limon Peel Powder ⁷ | | | | |
| Citric Acid ¹² | To pH 5.5-6.1 | | | |
| Water, Preservatives, | Q.S. to | Q.S. to | Q.S. to | Q.S. to |
| Perfume, and Optional | 100 | 100 | 100 | 100 |
| Components | | | | |

### The Examples in

Table 3 were phase stable, have a pH that prevents the surfactant from hydrolyzing (e.g. greater than or equal to 5.5), and have a consumer acceptable viscosity (e.g. greater than or equal to 2700 Pa). It is believed that these examples would be consumer preferred, as compared to the comparative examples in Table 2.

### The Examples in Table 4 and

Table 5 could be made and it is believed that these examples would be stable and have consumer acceptable viscosity. The Examples in Table 4 would have a total sodium chloride of 0.7% based on the specifications from suppliers and a ratio of polymer charge density to inorganic salt of 18:1.

TABLE 4

| | Ex. 5 | Ex. 6 | Ex. 7 |
|---|---|---|---|
| Lauramidopropyl Betaine ¹ | 9.75 | 9.75 | 9.75 |
| Sodium Cocoyl Isethionate ² | 6.00 | 6.00 | 6.00 |
| Sodium Lauryl Sarcosinate ³ | | | 4.00 |
| Polyquaternium 10 ⁴ | 0.55 | 0.55 | 0.55 |
| (JR-30M, CD 1.25 meq/g) | | | |
| Sclerotium Gum ^{5,6} | 0.50 | 0.50 | 0.50 |
| Sodium Benzoate ¹³ | 0.75 | 0.75 | 0.45 |
| Sodium Salicylate ¹⁴ | | 0.45 | 0.45 |
| Perfume | 1.00 | 1.00 | 1.00 |
| Citric Acid ¹² | To pH 5.5-6.5 | | |
| Water | QS to 100 | QS to 100 | QS to 100 |
| Number of Ingredients | 8 | 9 | 10 |
| Ratio of Anionic to | 0.6:1 | 0.6:1 | 1.0:1 |
| Amphoteric Surfactant | | | |

TABLE 5

| | Ex. 8 | Ex. 9 | Ex. 10 | Ex. 11 | Ex. 12 | Ex. 13 | Ex. 14 |
|---|---|---|---|---|---|---|---|
| Lauramidopropyl Betaine ¹ | 9.75 | 9.75 | | 5.36 | 2.44 | 2.44 | |
| Low Salt Cocamidopropyl | | | 9.75 | | | | |
| Betaine ¹⁵ | | | | | | | |
| Cocamidopropyl Betaine ¹⁶ | | | | 4.39 | 7.31 | 7.31 | 7.50 |
| Sodium Cocoyl Isethionate ² | 6.00 | 6.00 | 6.00 | 6.00 | 6.00 | 6.00 | 4.50 |
| Sodium Lauryl Sarcosinate ³ | 2.50 | 2.50 | 2.50 | 2.50 | 2.50 | 2.50 | |
| Polyquaternium 10 ⁴ | 0.55 | 0.55 | 0.55 | 0.55 | | | |
| (JR-30M, CD 1.25 meq/g) | | | | | | | |
| Polyquaternium 10 ¹⁹ | | | | | 0.10 | 0.40 | 0.25 |
| (KG-30M, CD 1.9 meq/g) | | | | | | | |
| Sclerotium Gum ^{5, 6} | 0.50 | 1.50 | 0.50 | 0.50 | 0.50 | 0.50 | 0.50 |
| Piroctone Olamine ¹⁷ | 0.50 | | | | | | |
| Zinc Pyrithione ¹⁸ | | 1.00 | | | | | |
| Water, Preservatives, pH | QS to | QS to | QS to | QS to | QS to | QS to | QS to |
| adjusters, Perfume, and | 100 | 100 | 100 | 100 | 100 | 100 | 100 |
| Optional Components | | | | | | | |
| Ratio of Anionic to | 0.9:1 | 0.9:1 | 0.9:1 | 0.9:1 | 0.9:1 | 0.9:1 | 0.6:1 |
| Amphoteric Surfactant | | | | | | | |
| Total Sodium Chloride | 0.07 | 0.07 | 0.06 | 0.81 | 1.3 | 1.3 | 1.3 |
| (including from surfactant) | | | | | | | |
| Ratio of Polymer Charge | 18:1 | 18:1 | 21:1 | 1.5:1 | 1.5:1 | 1.5:1 | 1.5:1 |
| Density to Inorganic Salt | | | | | | | |

### Suppliers for Examples in Table 2 to Table 5

1. Mackam® DAB-ULS available from Solvay®. Specification Range: Solids=34-36%, Sodium Chloride=0-0.5%. Average values are used for calculations: Actives=35%, Sodium Chloride=0.25%.
2. Hostapon® SCI-85 C available from Clariant®
3. SP Crodasinic™ LS30/NP MBAL available from Croda®
4. UCARE™ Polymer JR-30M available from Dow®
5. Sclerotium Gum, Amigum ER available from Alban Muller®
6. Sclerotium Gum, Actigum® CS 11 QD available from Cargill®
7. Sclerotium Gum and Citrus Limon Peel Powder, Fiber Design Sensation® available from Cargill®
8. Xanthan Gum, Keltrol® CG-SFT available from CP Kelco®
9. Carrageenan, GENUVISCO® Carrageenan CG-131 available from CP Kelco®
10. Konjac Gum, Nutricol® XP 3464 available from FMC Corporation
11. Gellan Gum, Kelcogel® CG-LA available from CP Kelco®
12. Citric Acid USP Anhydrous Fine Granular available from ADM®
13. Sodium Benzoate available from Kalama®
14. Sodium Salicylate available from JQC (Huayin) Pharmaceutical Co., Ltd
15. Dehyton® PK 45 from BASF® with Sodium Chloride removed, resulting in 33.05% Dry Residue, 0.21% Sodium Chloride, 32.84% Active used for calculations.
16. TEGO® Betain CK PH 12 available from Evonik®. Specification Range: Actives=28-32%, Sodium Chloride=4.5-6%. Average values are used for calculations: Actives=30%, Sodium Chloride=5.25%.
17. Octopirox® available from Clariant®
18. Zinc Pyrithione available from Lonza®
19. UCARE™ Polymer KG-30M available from Dow®

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

what is claimed is:

1. A stable shampoo composition comprising:
a. a surfactant system comprising:
i. about 3% to about 35% of an anionic surfactant;
ii. about 5% to about 15% of an amphoteric surfactant;
wherein the surfactant system is substantially free of sulphated surfactants;
b. about 0.01% to about 2% of a cationic polymer;
c. about 0.15% to about 1.5% of a thickener comprising sclerotium gum;
wherein the composition has a viscosity of greater than 2500 cP and a pH of greater than 5.5.

2. The stable shampoo composition of claim 1, further comprising less than about 1% inorganic salt.

3. The stable shampoo composition of claim 2, wherein the inorganic salt is selected from sodium chloride, potassium chloride, sodium sulfate, ammonium chloride, sodium bromide, and combinations thereof.

4. The stable shampoo composition of claim 1, wherein the stable shampoo composition has a viscosity of greater than 4000 cP.

5. The stable shampoo composition of claim 1, wherein the stable shampoo composition has a pH greater than 5.7.

6. The stable shampoo composition of claim 1, wherein the stable shampoo composition has a yield stress of greater than 0.003 Pa at a shear rate 10⁻² to 10⁻⁴ s⁻¹ according to the Herschel-Bulkley Test Method.

7. The stable shampoo composition of claim 1, wherein the stable shampoo composition has a ratio of polymer charge density to total inorganic salt of greater than or equal to 1.2:1.

8. The stable shampoo composition of claim 1, wherein the stable shampoo composition is substantially free of alkyl polyglucoside.

9. The stable shampoo composition of claim 1, wherein the stable shampoo composition is substantially free of fatty esters.

10. The stable shampoo composition of claim 1, wherein the shampoo stable shampoo composition has a % T at 600 nm of greater than 70%.

11. The stable shampoo composition of claim 1, wherein the shampoo stable shampoo composition lacks an in situ coacervate, as determined by the Microscopy Method to Determine Lack of In Situ Coacervate.

12. The stable shampoo composition of claim 1, wherein the anionic surfactant is selected from sodium, ammonium or potassium salts of isethionates, sodium, ammonium or potassium salts of sulfonates, sodium, ammonium or potassium salts of ether sulfonates, sodium, ammonium or potassium salts of sulfosuccinates, sodium, ammonium or potassium salts of sulfoacetates, sodium, ammonium or potassium salts of glycinates, sodium, ammonium or potassium salts of sarcosinates, sodium, ammonium or potassium salts of glutamates, sodium, ammonium or potassium salts of alaninates, sodium, ammonium or potassium salts of carboxylates, sodium, ammonium or potassium salts of taurates, sodium, ammonium or potassium salts of phosphate esters, and combinations thereof.

13. The stable shampoo composition of claim 1, wherein the cationic polymer is selected from cationic guars, cationic cellulose, cationic synthetic homopolymers, cationic synthetic copolymers, and combinations thereof.

14. The stable shampoo composition of claim 1, wherein the amphoteric surfactant is selected from betaines, sultaines, hydroxysultanes, amphohydroxypropyl sulfonates, alkyl amphoactates, alkyl amphodiacetates, and combination thereof.

15. The stable shampoo composition of claim 1, further comprising an antidandruff agent.

16. The stable shampoo composition of claim 15, wherein the antidandruff agent is selected from piroctone olamine, zinc pyrithione, and combinations thereof.

17. The stable shampoo composition of claim 1, wherein the stable shampoo composition is substantially free of silicones.

18. The stable shampoo composition of claim 1, further comprising 9 or fewer ingredients.

19. A method of making the shampoo stable shampoo composition of claim 1, wherein the sclerotium gum is added using high shear milling.
